(19) United States
(12) Patent Application Publication
(10) Pub. No.: US 2007/0249545 A1
(43) Pub. Date: Oct. 25, 2007

(54) DIHYDROFOLATE REDUCTASE INHIBITION BY EPIGALLOCATECHIN GALLATE COMPOUNDS

(76) Inventors: Jose Neptuno Rodriguez-Lopez, Murcia (ES); Encamacion Maria Navarro-Peran, Murcia (ES); Juan Cabezas-Herrera, Murcia (ES)

Correspondence Address: KLARQUIST SPARKMAN, LLP, 121 SW SALMON STREET, SUITE 1600, PORTLAND, OR, 97204, US

(21) Appl. No.: 11/574,216

(22) PCT Filed: Aug. 25, 2005
(86) PCT No.: PCT/IB05/03009

§ 371 Date: Feb. 23, 2007

(30) Foreign Application Priority Data
Aug. 27, 2004 (GB) 0419181.3
Nov. 12, 2004 (GB) 0425081.7

(51) Int. Cl. A61K 31/7048; A61K 31/353; G06F 19/00; C12P 17/06

(52) U.S. Cl. 514/27; 514/456; 435/125; 702/19

(57) ABSTRACT

This invention relates to the identification, production and use of Dihydrofolate Reductase (DHFR) inhibitors based on the green tea catechin Epigallocatechin Gallate (EGCG). These inhibitors are, for example, useful in therapy, in particular for the treatment of cancer, infection and inflammatory conditions.

This invention relates to methods and means for the development of novel anti-folate compounds useful in the treatment of cancer and other disorders.

Green tea catechins, which include (−)-epigallocatechin gallate (EGCG), (−)-epigallocatechin (EGC), (−)-epicatechin gallate (ECG), and (−)-epicatechin (EC), exhibit a range of biological activities¹ and comprise ca.15% of the dry weight of tea leaves, with EGCG being the most abundant. One cup (240 ml) of brewed green tea contains up to 200 mg EGCG.

The antioxidant, antibiotic and antiviral activities that have been attributed to EGCG² are increasingly used to promote green tea drinking. EGCG significantly lowers blood glucose and insulin levels and green tea extracts increase glucose metabolism in adipocytes⁴. Of particular interest, is the ability of tea catechins to inhibit tumour growth^{5,6}.

Green tea extracts have been shown in vitro to stimulate apoptosis and cell cycle arrest of various cancer cell lines, including prostate, lymphoma, colon, and lung¹. Moreover, EGCG inhibits tumour invasion and angiogenesis, processes that are essential for tumour growth and metastasis⁵.

The site of action and mechanism at the molecular level by which EGCG acts as an anti-carcinogen is poorly understood. EGCG has been implicated in the modulation of several transcription factors such as activator protein-1 (AP-1)⁷ and nuclear factor-kappaB (NF-κB)⁸, inhibition of gene expression such as tumor necrosis factor alpha (TNF-α)₉, vascular endothelial growth factor (VEGF)¹⁰ and nitric oxide synthase (NOS)⁸ and in the modulation of several cancer-related proteins that include urokinase, ornithine decarboxylase, matrix metalloproteinase and cyclooxygenase⁵. In addition, ester bond-containing tea polyphenols potently inhibit proteasome activity¹¹.

EGCG binds strongly to many biological molecules and affects a variety of enzyme activities and signal transduction pathways at concentrations from milli- to nano-molar¹². The effective concentration of EGCG in the blood or tissues of tea drinkers is in the range 0.1 to 1.0 μM¹², an important factor in deciding whether an in vitro modulation of biological activity by EGCG is likely to be relevant in vivo. Thus, although EGCG inhibits urokinase activity in vitro¹³, the concentration needed (2-10 mM), is at least 3 to 4 orders of magnitude higher than measured tissue/plasma levels of EGCG in vivo¹².

The present inventors have discovered that the green tea catechin (−)-epigallocatechin gallate (EGCG) is an anti-folate which inhibits the activity of dihydrofolate reductase (DHFR). Anti-folate compounds based on EGCG may be useful in the treatment of a range of disorders including cancer.

The invention, in various aspects, relates to methods and means for identifying and obtaining anti-folate compounds based on EGCG for use in therapy, in particular for the treatment of cancer.

One aspect of the invention provides a method of producing an anti-folate compound comprising;
providing an (−)-epigallocatechin gallate (EGCG) compound, and;
determining the interaction of said compound with DHFR.

EGCG compounds include both unmodified (−)-epigallocatechin gallate (EGCG) and modified forms of EGCG (i.e. modified EGCG compounds), for example, analogues, variants and derivatives of EGCG. Preferably, an EGCG compound comprises a gallate moiety, or a moiety with an analogous structure. A suitable gallate moiety may be ester bonded. In some embodiments, the EGCG compound may be a polyphenol, for example a flavanoid, such as a flavan-3-ol. EGCG compounds, including modified EGCG compounds are discussed in more detail below.

A method may further comprise;
modifying the structure of the EGCG compound, and;
determining the interaction of the modified EGCG compound with DHFR.

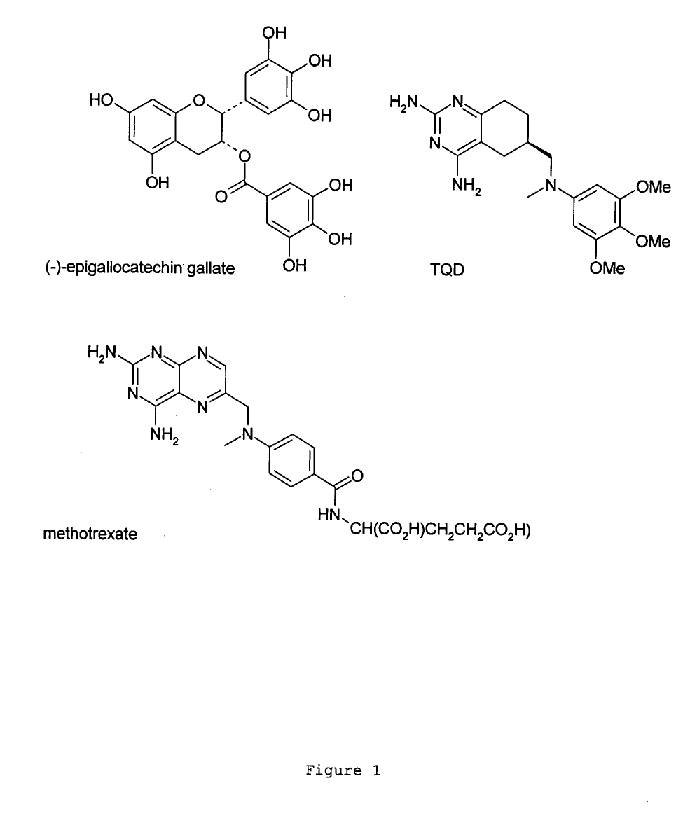

The structure of the EGCG compound may be modified to optimise the interaction of the compound with DHFR or to improve its pharmaceutical properties, for example to reduce side effects associated with the compound, increase the half-life of the compound in vivo, reduce the cost of synthesis of the compound, improve bio availability or increase the suitability of the compound for a particular method of administration. The modification of EGCG compounds, such as EGCG, is described in more detail below. In some preferred embodiments, the initial or starting EGCG compound for use in the present methods is EGCG. The structure of EGCG is shown in FIG. 1.

A method of producing an anti-folate compound may comprise;
modifying the structure of EGCG to produce a modified EGCG compound, and;
determining the interaction of said modified EGCG compound with DHFR.

The methods described above may be iterated in that an optimised or modified EGCG compound may itself be the basis for further optimisation and/or modification.

In some preferred embodiments, the interaction of an EGCG compound with DHFR may be determined in silico i.e. using computer-assisted techniques.

For example, a method for producing an anti-folate compound may comprise:
providing a structure comprising a three-dimensional representation of DHFR or a portion of DHFR;
providing an EGCG compound structure to be fitted to said DHFR structure or selected coordinates thereof
fitting the EGCG compound structure to said DHFR structure.

Fitting includes determining, by automatic or semi-automatic means, interactions between at least one atom of an EGCG compound molecular structure and at least one atom of a DHFR structure, and calculating the extent to which such an interaction is stable. Interactions include attraction and repulsion, brought about by charge, steric considerations and the like. Various computer-based methods for fitting are described further herein.

More specifically, the interaction of an EGCG compound with DHFR can be examined through the use of computer modelling using a docking program such as GOLD (Jones et al., J. Mol. Biol., 245, 43-53 (1995), Jones et al., J. Mol. Biol., 267, 727-748 (1997)), GRAMM (Vakser, I. A., Proteins, Suppl., 1:226-230 (1997)), DOCK (Kuntz et al, J. Mol. Biol. 1982, 161, 269-288, Makino et al, J. Comput. Chem. 1997, 18, 1812-1825), AUTODOCK (Goodsell et al, Proteins 1990, 8, 195-202, Morris et al, J. Comput. Chem. 1998, 19, 1639-1662.), FlexX, (Rarey et al, J. Mol. Biol. 1996, 261, 470-489), ICM (Abagyan et al, J. Comput. Chem. 1994, 15, 488-506), MCSS (Molecular Simulations, San Diego, Calif.), AUTODOCK (Scripps Research Institute, La Jolla, Calif.), Flo99 (Thistlesoft, Morris Township, N.J.), Ludi (Molecular Simulations, San Diego, Calif.), QUANTA (Molecular Simulations, San Diego, Calif.), Insight (Molecular Simulations, San Diego, Calif.), SYBYL (TRIPOS, Inc., St. Louis. Mo.) or LEAPFROG (TRIPOS, Inc., St. Louis, Mo.).

For example, an EGCG compound which is an analogue, variant, derivative or modified form of EGCG may be fitted by computer to the structure of DHFR to ascertain how well the shape and the chemical structure of the compound will bind to the DHFR. The interaction of a modified EGCG compound with DHFR may be determined relative to the interaction of EGCG with DHFR.

Also computer-assisted, manual examination of the EGCG binding site structure of DHFR may be performed. The use of programs such as GRID (Goodford, J. Med. Chem., 28, (1985), 849-857)—a program that determines probable interaction sites between molecules with various functional groups and an enzyme surface—may also be used to analyse the DHFR binding site to predict, for example, the types of modifications to the EGCG structure which will optimise binding.

Detailed structural information can be obtained about the binding of an EGCG compound to DHFR using the methods outlined above, and in the light of this information, adjustments can be made to the structure or functionality of the compound, e.g. to alter its interaction with DHFR. The above steps may be repeated and re-repeated as necessary.

For example, a method may further comprise the step of modifying or optimising the structure of an EGCG compound. In particular, the structure of the EGCG compound may be modified to optimise binding to the DHFR structure. The modified EGCG compound may be fitted to the DHFR structure or selected coordinates thereof.

A method for producing an anti-folate compound may comprise:
providing a structure comprising a three-dimensional representation of DHFR or a portion of DHFR;
fitting a starting EGCG compound structure to the DHFR structure or selected coordinates thereof;
modifying the EGCG structure to optimise the interaction with the DHFR structure and;
fitting the modified EGCG structure to the DHFR structure.

The starting EGCG compound structure may be EGCG.

A structure may be optimised by making modifications to the structure, for example, by adding molecular scaffolding, adding or varying functional groups, or connecting the molecule with other molecules (e.g. using a fragment linking approach) such that the chemical structure of the modulator molecule is changed while its original modulating functionality is maintained or enhanced. Such optimisation is regularly undertaken during drug development programmes to e.g. enhance potency, promote pharmacological acceptability, increase chemical stability etc. of lead compounds. For example, the ester bond present in EGCG may be chemically modified such that the modified compound is not susceptible to hydrolysis, for example by esterases. This may increase the half-life of the compound in vivo.

Modifications to the EGCG compound structure will be those conventional in the art known to the skilled medicinal chemist, and will include, for example, substitutions or removal of groups containing residues which interact with the amino acid side chain groups of a DHFR structure. For example, the replacements may include the addition or removal of groups in order to decrease or increase the charge of a group in a test compound, the replacement of a charge group with a group of the opposite charge, or the replacement of a hydrophobic group with a hydrophilic group or vice versa.

For example, comparison with a range of other DHFR structures containing folate or various inhibitors shows that the non-ester trihydroxybenzene moiety lies outside the consensual substrate/inhibitor envelope. In order to accommodate this ring, the Leu-22 side chain of DHFR is required to adopt a different orientation. Removal or replacement of the non-ester trihydroxybenzene moiety may improve the interaction between the modified EGCG compound and DHFR.

Another modification to the EGCG compound structure, for example, may be the methylation of the hydroxyl groups of the ester bonded gallate moiety, for example to reduce or prevent further auto oxidation of the compound. This modification may increase the bioavailability of the effective drug form.

Another modification may be the introduction of a hetero atom instead of the carbon atom between the two hydroxyl groups of the catechol ring of the EGCG compound structure. This may improve hydrogen bond formation between the modified compound and the active centre of DHFR.

In some embodiments, a template molecule may be selected onto which chemical groups that mimic the EGCG pharmacophore can be grafted. The template molecule and the chemical groups grafted on to it can conveniently be selected so that the modified EGCG compound is easy to synthesise, is likely to be pharmacologically acceptable, and does not degrade in vivo, while retaining the biological activity of EGCG.

A modification may, for example, include the addition or substitution of one or more atoms or groups in the EGCG structure with one or more of hydrogen; an optionally substituted C₁₋₇ alkyl group; a C₃₋₂₀ heterocyclyl group; a C₅₋₂₀ aryl group; an optionally substituted heterocyclic ring having from 4 to 8 ring atoms; or one or more of the following substituent groups: Halo: —F, —Cl, —Br, and —I; Hydroxy: —OH; Ether: —OR, C₁₋₇ alkoxy: —OR, wherein R is a C₁₋₇ alkyl group; C₁₋₂ alkdioxylene; Oxo (keto, -one): ═O; Imino (imine): ═NR; Formyl: —C(═O)H; Acyl (keto): —C(═O)R; Ester: —C(═O)OR; Acyloxy: —OC(═O)R; Amido: —C(═O)NR¹R²; Acylamido: —NR¹C(═O)R²; Thioamido: —C(═S)NR¹R²; Tetrazolyl; Amino: —NR¹R²; Amidine: —C(═NR)NR₂; Nitro: —NO₂; Nitroso: —NO; Azido: —N₃; Cyano: —CN; Isocyano: —NC; Cyanato: —OCN; Isocyanato: —NCO; Thiocyano: —SCN; Isothiocyano (isothiocyanato): —NCS; Sulfhydryl: —SH; Thioether: —SR; Disulfide: —SS—R; Sulfone: —S(═O)₂R; Sulfine: —S(═O)R; Sulfonyloxy: —OS(═O)₂R; Sulfinyloxy: —OS(═O)R; Sulfamino: —NR¹S(═O)₂OH; Sulfonamino: —NR¹S(═O)₂R; Sulfinamino: —NR¹S(═O)R, Sulfamyl: —S(═O)NR¹R²; Phosphoramidite: —OP(OR¹)—NR²₂; and Phosphoramidate: —OP(═O)(OR¹)—NR²₂.

C₁₋₇ alkyl is a monovalent moiety obtained by removing a hydrogen atom from a C₁₋₇ hydrocarbon compound having from 1 to 7 carbon atoms, which may be aliphatic or alicyclic, or a combination thereof, and which may be saturated, partially unsaturated, or fully unsaturated.

C₃₋₂₀ heterocyclyl is a monovalent moiety obtained by removing a hydrogen atom from a ring atom of a C₃₋₂₀ heterocyclic compound, said compound having one ring, or two or more rings (e.g., spiro, fused, bridged), and having from 3 to 20 ring atoms, atoms, of which from 1 to 10 are ring heteroatoms, and wherein at least one of said ring(s) is a heterocyclic ring. Preferably, each ring has from 3 to 7 ring atoms, of which from 1 to 4 are ring heteroatoms. “C₃₋₂₀” denotes ring atoms, whether carbon atoms or heteroatoms.

C₅₋₂₀ aryl is a monovalent moiety obtained by removing a hydrogen atom from an aromatic ring atom of a C₅₋₂₀ aromatic compound, said compound having one ring, or two or more rings (e.g. fused), and having from 5 to 20 ring atoms, and wherein at least one of said ring(s) is an aromatic ring. Preferably, each ring has from 5 to 7 ring atoms. The ring atoms may be all carbon atoms or the ring atoms may include one or more heteroatoms, for example oxygen, nitrogen, and sulphur.

In many cases, substituents may themselves be substituted. For example a C₁₋₇ alkyl group, a C₃₋₂₀ heterocyclyl group, a C₅₋₂₀ aryl group, or heterocyclic ring as described above may comprise one or more substituent groups.

It will be understood that these are only examples of the type of substitutions considered by medicinal chemists in the development of new pharmaceutical compounds and other modifications may be made, depending upon the nature of the starting compound and its activity.

The structure of EGCG compounds may be modelled according to their physical properties, e.g. stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can be used in this modelling process.

In some embodiments, modifying an EGCG compound may comprise creating a new chemical compound based on EGCG, for example by modelling the pharmacophore as described above; searching databases of libraries of known compounds for EGCG derivatives or analogues (e.g. an EGCG compound which is listed in a computational screening database containing three dimensional structures of known compounds); or simulating EGCG compounds having substitute moieties or certain structural features.

In some embodiments, modifying may include computational screening of one or more databases of compounds in which the three dimensional structure of the compound is known, with the structure of EGCG to identify a modified EGCG compound and interacting the modified EGCG compound (e.g., docking, aligning, matching, interfacing) with the three dimensional structure of DHFR protein by computer (e.g. as described by Humblet and Dunbar, Animal Reports in Medicinal Chemistry, vol. 28, pp. 275-283, 1993, M Venuti, ed., Academic Press).

Modification of the structure in silico allows predictions to be made as to how the modified structure interacts with the DHFR. Preferably, modification provides an EGCG compound structure that is predicted to have a conformation compatible with the EGCG binding site on the structure of the DHFR.

A method may further comprise identifying a modified EGCG compound structure that is predicted to bind to the DHFR protein with improved or optimised properties.

Improved binding properties may include decreased inhibition constants (Ki or Ki*) or dissociation constants relative to EGCG and/or changes to the type of inhibition (e.g. competitive, slow tight-binding, irreversible).

The above-described processes may be iterated in that the optimised or modified EGCG compound may itself be the basis for further compound design.

DHFR suitable for use in the methods described herein may include any eukaryotic or prokarytic DHFR, and may for example be a vertebrate DHFR, including an avian DHFR such as chicken DHFR or a mammalian DHFR such as bovine DHFR, non-human primate DHFR or human DHFR.

The sequences and structures of a range of DHFR proteins are available in the art. Crystal structures of DHFR proteins in the Protein Data Bank include DHFR from: Candida Albicans, Lactobacillus Casei, Pneumocystis Carinii, Thermotoga Maritima, Mycobacterium Tuberculosis, chicken liver, Escherichia coli, Plasmodium Falciparum, Bacteriophage T4 and human.

DHFR may be used in the presence of a cofactor such as NADPH.

In some embodiments, the structure which comprises a three-dimensional representation of DHFR or a portion of DHFR may further comprise a cofactor such as NADPH and/or an anti-folate compound, such as tetrahydroquinazoline (TQD), methotrexate, trimetrexate, folate, 5-deazafolate and furopyrimdine. Suitable three-dimensional structures of complexes of DHFR bound to antifolate compounds may include PDB acc code 1S3V, PDB acc code 1AO8, PDB acc code 1BZF, PDB acc code 1DHF and PDB acc code 1HFQ.

It is not generally necessary to interact or fit an EGCG compound structure to each residue in the DHFR binding site. Suitable EGCG compounds may be fitted with a subset of residues described for the DHFR binding site. An EGCG compound may, for example, comprise a conformation that promotes the formation of covalent or non-covalent cross-linking between the target site and the candidate chemical compound.

Where a potential modified compound has been developed by fitting a starting EGCG compound to the DHFR structure of the invention and predicting from this a modified EGCG compound with improved binding properties, a method may further include the step of obtaining or synthesizing the modified EGCG compound

A computer-assisted method of structure based drug design of an anti-folate compound may comprise: (a) providing a three dimensional structure of DHFR with a starting EGCG compound; (b) designing a modified EGCG compound using the three-dimensional structure or model; and (c) chemically synthesizing the modified EGCG compound.

Methods to synthesize suitable chemical compounds are known to those of skill in the art and depend upon the structure of the chemical being synthesized. Methods to evaluate the bioactivity of the synthesized compound depend upon the bioactivity of the compound (e.g. inhibitory or stimulatory) and are disclosed herein.

A synthesised EGCG compound may be evaluated or tested in an in vivo or in vitro biological system in order to determine its activity and/or its pharmaceutical or pharmacological properties. Further optimisation or modification can then be carried out to arrive at one or more final compounds or in vivo or clinical testing.

For example, the ability of said test compound to inhibit DHFR activity may be tested by contacting said compound with DHFR protein to determine the ability of said compound to interact with or inhibit DHFR.

In some embodiments, the compound may be contacted with DHFR under conditions suitable to determine its activity. For example, the EGCG compound may contacted with DHFR in the presence of NADPH, and typically a buffer and 7,8-dihydrofolate (DHF) substrate, to determine the ability of said EGCG compound to inhibit DHFR. So, for example, an assay mixture for DHFR may be produced which comprises NADPH, 7,8-dihydrofolate (DHF) substrate and buffer.

The kinetic properties of DHFR inhibition may be measured. For example, Ki or Ki* of the inhibition may be determined.

In other embodiments, a method may further include the step of obtaining or synthesizing the EGCG compound, forming a complex of a DHFR protein and said compound; said complex diffracting X-rays for the determination of atomic coordinates of said complex; and analysing the complex by X-ray crystallography to determine the ability of said compound to interact with the DHFR.

X-ray diffraction data can be collected by a variety of means, once a crystal or crystal complex is grown, in order to obtain the atomic coordinates of the crystallized molecule or molecular complex. With the aid of specifically designed computer software, such crystallographic data can be used to generate a three dimensional structure of the molecule or molecular complex. Various methods used to generate and refine the three dimensional structure of a crystallized molecule or molecular structure are well known to those skilled in the art, and include, without limitation, multiwavelength anomalous dispersion (MAD), multiple isomorphous replacement, reciprocal space solvent flattening, molecular replacement, and single isomorphous replacement with anomalous scattering (SIRAS).

In some embodiments, the interaction of an EGCG compound with DHFR may be determined in vitro, without computer assisted modelling techniques.

For example, interaction may be determined by determining the binding of the EGCG compound to DHFR.

A method of producing an anti-folate compound may comprise; contacting an EGCG compound with DHFR and; determining the binding of the EGCG compound to DHFR.

The presence of binding is indicative that the EGCG compound is an anti-folate.

An EGCG compound may be part of a library of EGCG compounds, produced, for example, by combinatorial chemistry techniques. A library of EGCG compounds may be contacted with DHFR and the binding of one or more members of the library to DHFR determined.

Binding may, in some embodiments, be determined relative to the binding of EGCG to DHFR.

Suitable methods of determining binding are well known in the art.

A method may comprise determining the ability of an EGCG compound to inhibit DHFR activity. For example, a method of producing an anti-folate compound may comprise:

contacting an EGCG compound with DHFR and;

determining the inhibition of DHFR activity by the EGCG compound.

Another aspect of the invention provides a method of determining the optimal physiological dose of an EGCG compound for inhibiting growth of a cancer cell may comprise the steps of:
determining the inhibition of DHFR in a cancer cell by progressively increasing doses of the EGCG compound;
determining the viability of a non-cancer cell in the presence of said doses; and
selecting the dose of the EGCG compound that achieves DHFR inhibition in the cancer cell with continued viability of the non-cancer cell.

The optimal physiological dose of the EGCG compound can be determined by monitoring the dose response curve of DHFR inhibition in response to the addition of various doses of the EGCG compound. Such a method can be performed in vitro and/or in vivo.

The EGCG compound for use in the above method may be obtained from an extract of green tea or may be a synthetic EGCG compound.

The dose of the EGCG compound used in the above method can be prepared as a pharmaceutical composition by formulating the dose of the EGCG compound with a pharmaceutically acceptable carrier, adjuvant or excipient. Such a pharmaceutical composition can be further formulated with an agent which modulates intracellular pH. Examples of said agents are discussed below.

Suitable EGCG compounds include EGCG and modified EGCG compounds as described herein.

In other embodiments, the binding of an EGCG compound to DHFR in the presence of a test compound may be assayed by contacting the EGCG compound and the test compound with DHFR, and determining the binding of the EGCG compound to DHFR in the presence of the test compound.

Displacement of the binding of the EGCG compound to DHFR may be indicative that the test compound is a putative anti-folate. In a preferred embodiment, the EGCG compound may be radiolabelled. More preferably, the EGCG compound is radiolabelled EGCG.

Suitable methods for performing competition or displacement assays are well known in the art.

Test compounds having a putative anti-folate activity as determined by the above method may be useful in the formulation of pharmaceutical compositions and drugs.

An EGCG compound which binds to DHFR and/or inhibits DHFR activity may be identified as a putative anti-folate compound. The identified compound may be modified or optimised, for example to enhance binding or other pharmacological properties. Optimisation and/or modification may be performed by an in silico or in vitro method as described herein.

A method of producing an anti-cancer compound may comprise;
modifying an EGCG compound,
contacting the modified EGCG compound with DHFR; and,
determining one or both of: the binding of the modified EGCG compound to DHFR and the activity of DHFR in the presence of the modified EGCG compound.

Modifying EGCG compounds are described in more detail above.

Activity may be determined in the presence and absence of modified EGCG compound. A decrease in activity in the presence relative to the absence of test compound is indicative that the modified EGCG compound inhibits DHFR.

In some embodiments, inhibition of DHFR may be determined relative to the inhibition of DHFR by EGCG.

The skilled person is readily able to determine and measure DHFR activity, for example by the depletion of substrate (e.g. NADPH or 7,8-dihydrofolate (DHF)) or the formation of product (e.g. 5,6,7,8-tetrahydrofolate (THF)).

A modified EGCG compound which is identified as described herein may be used in a secondary screen, for example in a cell or animal model, in order to determine one or more biological effects of the compound.

The modified EGCG compound may be contacted with a mammalian cell line, in particular a cancer cell line, and the effect of the compound on the cell determined. The effect of the modified EGCG compound may be determined relative to the effect of EGCG or other DHFR inhibitor.

The modified EGCG compound may be contacted with the cell in the presence of a pH modulator so as to achieve an intracellular pH in the presence of said EGCG of at least about pH 6.5.

The effect of the EGCG compound in vitro on apoptosis and/or cell cycle arrest in a cancer cell line, such as a prostate, lymphoma, colon, or lung cancer cell line, may be determined. Apoptosis and/or cell cycle arrest in a cancer cell line may be determined relative to a non-cancer cell line.

The effect of the EGCG compound on the expression of one or more biomarkers may be determined. Suitable biomarkers may include transcription factors such as activator protein-1 (AP-1) and nuclear factor-kappaB (NF-κB), tumor necrosis factor alpha (TNF-α), vascular endothelial growth factor (VEGF) and nitric oxide synthase (NOS) and cancer-related proteins such as urokinase, ornithine decarboxylase, matrix metalloproteinase and cyclooxygenase, and proteasome activity.

The effect of the EGCG compound on tumour invasion and angiogenesis may be determined in a non-human animal model, for example a non-human animal xenograft model. The effect of the modified EGCG compound may be determined relative to the effect of EGCG or other DHFR inhibitor.

Other biological effects may be determined in a non-human animal model. For example, side effects and toxicology and/or other pharmaceutical parameters, such as uptake and in vivo half-life, may be evaluated.

Another aspect of the invention provides a modified EGCG compound which inhibits DHFR and which is identified or produced by the methods described herein.

Another aspect of the invention provides the use of an EGCG compound obtained by a method described herein compound in the manufacture of a medicament for the treatment of a disease condition and a method of treating a disease condition in an individual comprising administering an EGCG compound obtained by a method described herein.

Suitable disease conditions include cancer, atherosclerotic conditions, pathogen infection and inflammatory disorders such as psoriasis, rheumatoid arthritis and Crohn's disease.

Cancer includes all types of solid cancers and malignant lymphomas and especially leukaemia, skin cancer, bladder cancer, breast cancer, uterus cancer, ovary cancer, prostate cancer, lung cancer, colon cancer, pancreas cancer, renal cancer, stomach cancer and cerebral cancer.

EGCG compounds as described herein may be useful in the formulation of pharmaceutical compositions and drugs.

A method for preparing a medicament, pharmaceutical composition or drug, for example for use in the treatment of cancer may comprise:
(a) identifying or producing a modified EGCG compound by a method described herein;
(b) optimising the structure of the EGCG compound; and
(c) preparing a medicament, pharmaceutical composition or drug containing the optimised compound.

A method for preparing an anti-folate medicament, pharmaceutical composition or drug, may comprise:

(a) modifying the structure of EGCG to optimise its binding to DHFR; and,

(b) preparing a medicament, pharmaceutical composition or drug containing the optimised EGCG compound.

Whilst an EGCG compound may be administered alone, it is preferable to present it as a pharmaceutical composition (e.g. formulation) which comprises the EGCG compound, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Methods of the invention may therefore comprise the step of formulating an EGCG compound with a pharmaceutically acceptable carrier, adjuvant or excipient.

A method of producing a pharmaceutical composition may comprise;
admixing an EGCG compound obtained by a method described herein with a with a pharmaceutically acceptable carrier, adjuvant or excipient.

In some applications a pharmaceutical composition may consist of an EGCG compound and a pharmaceutically acceptable carrier, adjuvant or excipient. Suitable EGCG compounds include EGCG and modified EGCG compounds as described herein.

For example, a sufficient dose of a pharmaceutical composition consisting of an EGCG compound and a pharmaceutically acceptable carrier, adjuvant or excipient may be administered to a patient to achieve a concentration of 0.1 to 1.0 μM of the EGCG compound in the blood or tissues of the patient. Such a concentration of the EGCG compound may be optimal for DHFR inhibition.

Suitable EGCG compounds include EGCG and modified EGCG compounds as described above.

In some embodiments, EGCG compounds as described herein may also be used in conditions in which cellular levels of folic acid are low. Low cellular levels of folic acid may be achieved by a diet low in folic acid or by administration of an agent that decreases folic acid intestinal absorption or decreases the transport of folic acid to cells.

Therefore, another aspect of the invention provides a pharmaceutical composition comprising an EGCG compound in combination with an agent that decreases folic acid intestinal absorption or decreases the transport of folic acid to cells.

A method of making a composition may comprise, for example, admixing an EGCG compound with an agent that decreases folic acid intestinal absorption or decreases the transport of folic acid to cells and a pharmaceutically acceptable carrier, adjuvant or excipient.

Other aspects of the invention provide the use of an EGCG compound in combination with an agent that decreases folic acid intestinal absorption or decreases the transport of folic acid to cells in the manufacture of a medicament for use in the treatment of a disease condition and a method of treating a disease condition comprising administering an EGCG compound in combination with an agent that decreases folic acid intestinal absorption or decreases the transport of folic acid to cells.

Suitable EGCG compounds include EGCG and modified EGCG compounds as described above.

Suitable agents that decrease folic acid intestinal absorption or decrease the transport of folic acid to cells include antacids e.g. omeprazole and ranitidine, and sulfasalazine.

Suitable disease conditions are described above.

The interaction between EGCG and DHFR is modulated by pH. EGCG has only a single phenolic OH group available for hydrogen bonding to Glu-30 (O . . . O distance 2.7 Å). pH modulates this interaction with a pKa ca. 6.5. At acidic pHs, the interaction of EGCG with DHFR is reduced. Modulation of pH may be useful in the therapeutic application of EGCG compounds.

Another aspect of the invention provides a pharmaceutical composition comprising an EGCG compound and an agent which modulates intracellular pH.

A method of making a composition may comprise, for example, admixing an EGCG compound, an agent that modulates intracellular pH and a pharmaceutically acceptable carrier, adjuvant or excipient.

Other aspects of the invention provide the use of an EGCG compound and an agent which modulates intracellular pH in the manufacture of a medicament for use in the treatment of a disease condition and a method of treating a disease condition comprising administering an EGCG compound and an agent which modulates intracellular pH.

Suitable EGCG compounds include EGCG and modified EGCG compounds as described above.

In some embodiments, the agent which modulates intracellular pH may increase the intracellular pH of prokaryotic cells. For example, the agent may be a proton pump inhibitor, such as omeprazole, lansoprazole, pantoprazole, rabeprazole or esomeprazole.

These agents may be useful in combination with an EGCG compound in the treatment of disease conditions such as chronic gastritis, peptic ulceration, gastric cancer, and Helicobacter pylori infection.

In other embodiments, the agent which modulates intracellular pH may increase the intracellular pH of eukaryotic cancer cells. Suitable agents include cesium, rubidium or potassium salts, in particular chloride salts.

These agents may be particularly useful in combination with an EGCG compound in the treatment of disease conditions such as cancer.

Other therapies which lead to a rise in the intracellular pH of cancer cells may be used in combination with EGCG compounds. For example, a method of treating a cancer cell comprising treating the cancer cell to increase the intracellular pH of said cancer cell and administering an EGCG compound in an effective amount to inhibit DHFR in said cell

Bacteria synthesise folic acid. The anti-bacterial effect of an EGCG compound may be increased by agents which decrease folic acid levels in the bacteria. This is exemplified herein using EGCG and sulfamethoxazole.

Another aspect of the invention provides a pharmaceutical composition comprising an EGCG compound and an agent which inhibits bacterial folic acid synthesis.

A method of making a composition may comprise, for example, admixing an EGCG compound, an agent which inhibits bacterial folic acid synthesis and a pharmaceutically acceptable carrier, adjuvant or excipient.

Other aspects of the invention provide the use of an EGCG compound and an agent which inhibits bacterial folic acid synthesis in the manufacture of a medicament for use in the treatment of a disease condition and a method of treating a disease condition comprising administering an EGCG compound and an agent which inhibits bacterial folic acid synthesis.

Suitable EGCG compounds include EGCG and modified EGCG compounds as described above.

Suitable agents which inhibit bacterial folic acid synthesis include inhibitors of the incorporation of p-aminobenzoic acid, such as amides of sulfonic acids, which include sulfonamides (sulfamethoxazole, sulfadiazine, and sulfadoxine) and sulfones (dapsone and sulfonylbisformanilide).

Preferably, the sulfonic acid amide and the EGCG compound are administered so as to produce a 20:1 ratio of sulphonamide to EGCG compound in blood and tissues of an individual, or formulated to produce such a ratio, upon administration.

For example, the sulfonic acid amide and the EGCG compound may be formulated in the ratio of 5:1 of sulfonic acid amide to EGCG compound.

Disease conditions suitable for treatment according to these embodiments include microbial infections, for example bacterial infections, or conditions associated therewith.

Bacterial infections may include infection by any gram positive or gram-negative organism including Stenotrophomonas maltophilia.

Other conditions associated with infection that may be suitable for treatment include Pneumocystis carinii pneumonia, typhoid fever, shigellosis, enterotoxigenic Escherichia coli diarrhoea, Nocardia infection, otitis media, and chronic bronchitis.

EGCG compounds described herein may also be useful in increasing the efficacy of other anti-folates.

Another aspect of the invention provides a pharmaceutical composition comprising an EGCG compound and an anti-folate compound.

A method of making a composition may comprise, for example, admixing an EGCG compound, an anti-folate compound and a pharmaceutically acceptable carrier, adjuvant or excipient.

Other aspects of the invention provide the use of an EGCG compound and an anti-folate compound in the manufacture of a medicament for use in the treatment of a disease condition and a method of treating a disease condition comprising administering an EGCG compound and an anti-folate compound.

For example, a method for enhancing the efficacy of an anti-folate treatment may comprise co-administering the anti-folate compound with an effective amount of an EGCG compound to achieve inhibition of DHFR.

Suitable EGCG compounds include EGCG and modified EGCG compounds as described above.

Anti-folate compounds may include methotrexate, aminopterine, trimethoprim, diaveridine, pymethamine, tetroxoprim, pitrexim, cotrimoxazole and trimetrexate.

Disease conditions suitable for treatment are described above and include cancer, atherosclerotic conditions, pathogen infection, and inflammatory disorders such as psoriasis, rheumatoid arthritis and Crohn's disease, as described above. The pharmaceutical composition of the invention may also be used as an immunosuppressive following organ transplant.

EGCG compounds may also be useful in the treatment of infection in individuals with a poor tolerance of conventional anti-folates such as trimethoprim (TMP), for example, individuals with reduced or impaired poor renal function.

Anti-folates may provoke severe skin reactions, bone marrow suppression, and thrombocytopenia in such individuals. Aspects of the invention provide the use of an EGCG compound in the manufacture of a medicament for use in the treatment of bacterial infection in a individual with low tolerance to an anti-folate such as trimethoprim (TMP), and a method of treating a bacterial infection in an individual with low tolerance to an anti-folate, such as trimethoprim (TMP), comprising administering an EGCG compound.

A composition for use in treating a bacterial infection in an individual with low tolerance to an anti-folate, such as trimethoprim (TMP), may be produced by:
admixing an EGCG compound and a pharmaceutically acceptable excipient.

A bacterial infection may include an infection by any gram-positive or gram-negative bacterial pathogen, including Stenotrophomonas maltophilia infection.

The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. The precise nature of the carrier or other material will depend on the route of administration, which may be oral, or by injection, e.g. cutaneous, subcutaneous or intravenous.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, or Lactated Ringer's Injection. Preservatives, stabilisers, buffers, antioxidants and/or other additives may be included, as required.

Suitable carriers, excipients, etc. can be found in standard pharmaceutical texts, for example, Remington's Pharmaceutical Sciences, 18th edition, Mack Publishing Company, Easton, Pa., 1990.

The pharmaceutical compositions and formulations may conveniently be presented in unit dosage form and may be prepared by any methods well known in the art of pharmacy. Such methods include the step of bringing into association the EGCG compound with the carrier which constitutes one or more accessory ingredients. In general, the compositions are prepared by uniformly and intimately bringing into association the active compound with liquid carriers or finely divided solid carriers or both, and then if necessary shaping the product.

Other aspects of the invention relate to compositions and products for preventing or reducing susceptibility to ill-health, for example nutritional food products or food supplements.

A nutritional food product may comprise a modified EGCG compound as described herein.

Other food products may comprise green tea catechins, such as EGCG, or modified EGCG compounds, with a depleted folic acid content or alternatively, green tea catechins in combination with a vitamin supplement other than vitamin B12.

Various further aspects and embodiments of the present invention will be apparent to those skilled in the art in view of the present disclosure. All documents mentioned in this specification are incorporated herein by reference in their entirety.

The invention encompasses each and every combination and sub-combination of the features that are described above.

Certain aspects and embodiments of the invention will now be illustrated by way of example and with reference to the figures described above and tables described below.

FIG. 1 shows the structural formulae of (−)-epigallocatechin gallate (EGCG), (R)-6-{[methyl-(3,4,5-trimethoxyphenyl)amino]methyl}-5,6,7,8-tetrahydroquinazoline-2,4-diamine (TQD) and methotrexate.

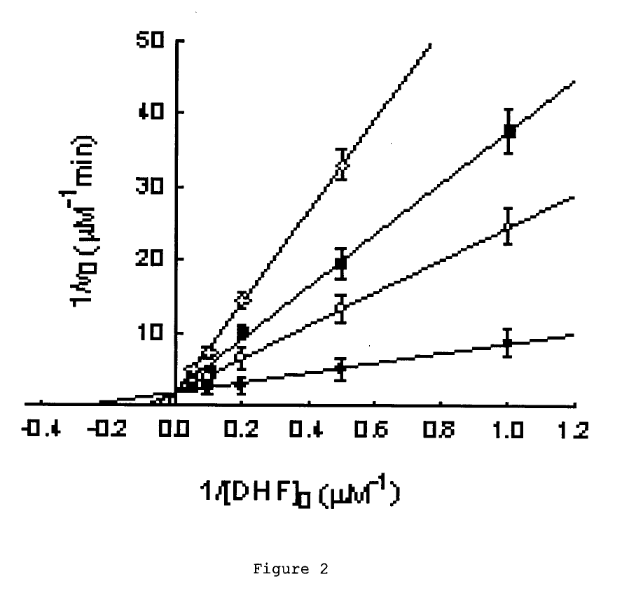

FIG. 2 shows Lineweaver-Burk plots of the reaction of chicken liver DHFR (3.3 nM) with DHF and NADPH. EGCG concentrations were 0 μM (closed circles), 25 μM (open circles), 50 μM (closed squares), and 100 μM (open diamonds). Each point represents the mean±s.d. of five separate experiments.

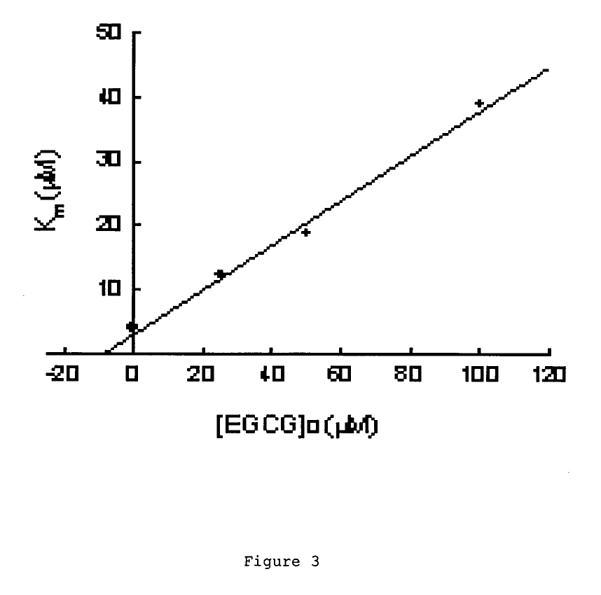

FIG. 3 shows secondary plots for the apparent Michaelis constant of DHFR for dihydrofolate (K_{DHF}), obtained from FIG. 3, versus the concentration of EGCG.

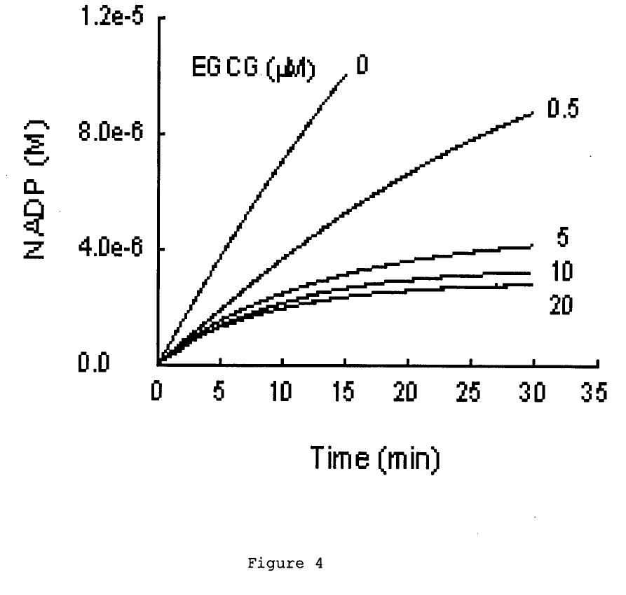

FIG. 4 shows progress curves for the slow, tight-binding inhibition of bovine liver DHFR by EGCG.

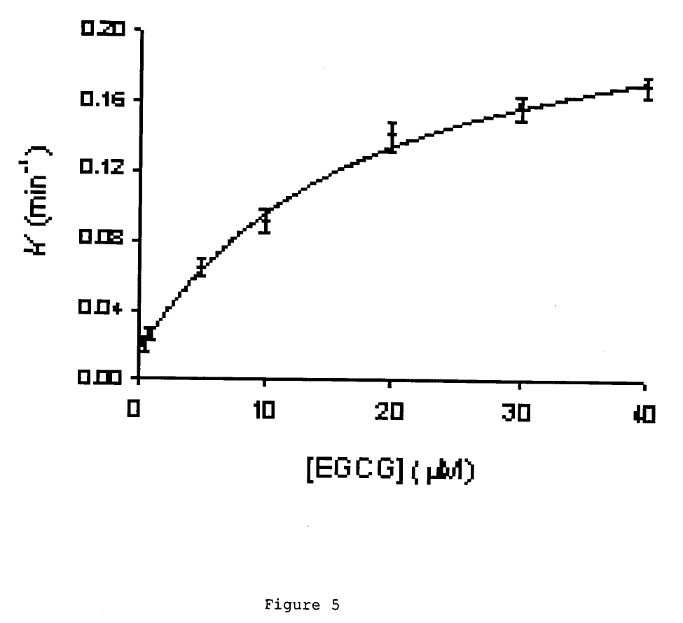

FIG. 5 shows non-linear regression analysis of the progress curves presented in FIG. 5 to Eqn. 1

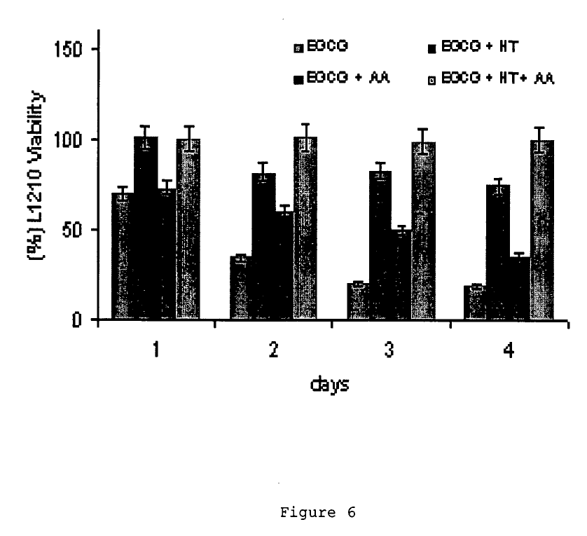

FIG. 6 shows time-dependent loss of L1210 viability induced by 20 μM EGCG. Data shown are expressed as a percentage of untreated control cells and represents the mean values±s.d. determined from three independent experiments.

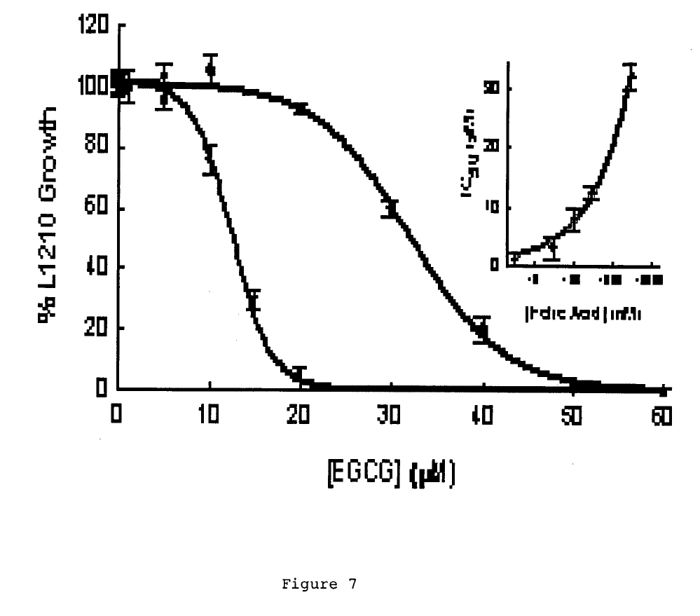

FIG. 7 shows the effect of different EGCG concentrations on L1210 cell growth after 29 h of treatment at two different folic acid concentrations. The inset shows the dependence of the IC₅₀ values on the folic acid concentration added to the medium.

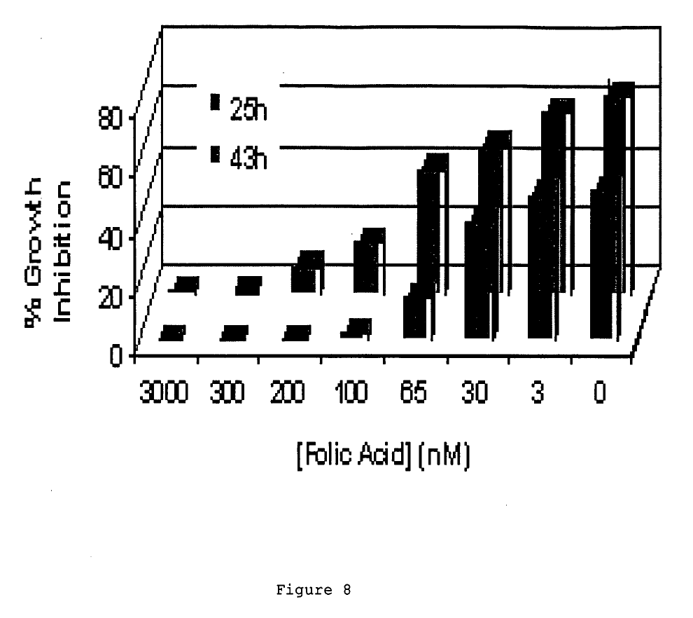

FIG. 8 shows the growth-inhibitory effects of 10 μM EGCG at various folic acid concentrations at two different time intervals.

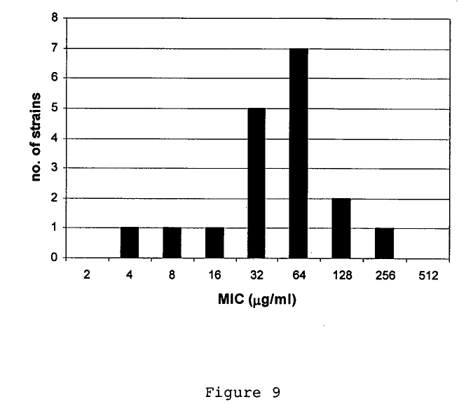

FIG. 9 shows the number of S. maltophilia strains (n=18) inhibited at each EGCG concentration tested.

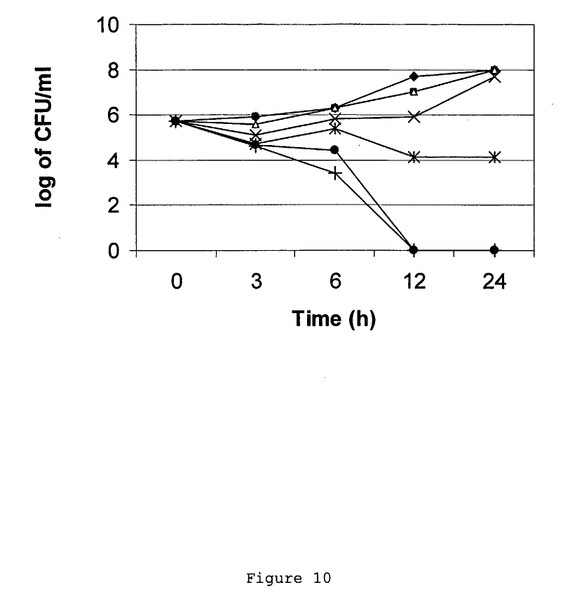

FIG. 10 shows the effect of EGCG on S. maltophilia strain 1 viability in liquid medium (time kill curve). S. maltophilia strain 1 was cultured aerobically in cation adjusted Mueller-Hinton broth at 37° C. with reciprocation in the presence of EGCG at concentrations of 512 (+), 256 (●), 128 (*), 64 (x), 32 (Δ), 16 (▪), and 0 (♦) μg/ml. Culture samples (100 μl) were taken at the times indicated and viability was measured by the plate colony count technique.

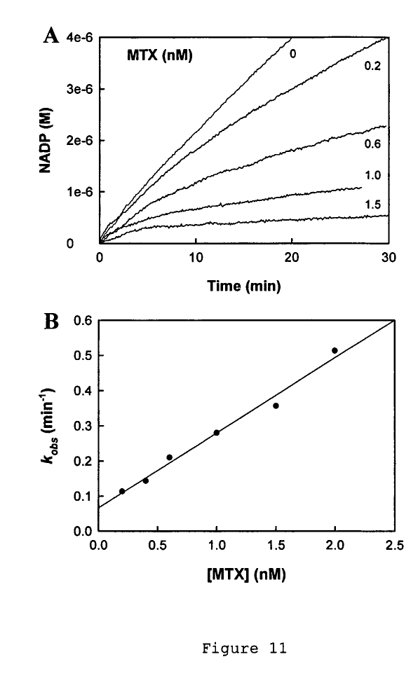

FIG. 11 shows the inhibition of S. maltophilia DHFR activity by MTX at pH 8.0. (A) Reaction progress curves at inhibitor concentrations of 0, 0.2, 0.6, 1.0, and 1.5 nM. (B) Replot of k_{obs} versus MTX concentration. k_{obs} is derived from the progress curves as described in Eqn. 1.

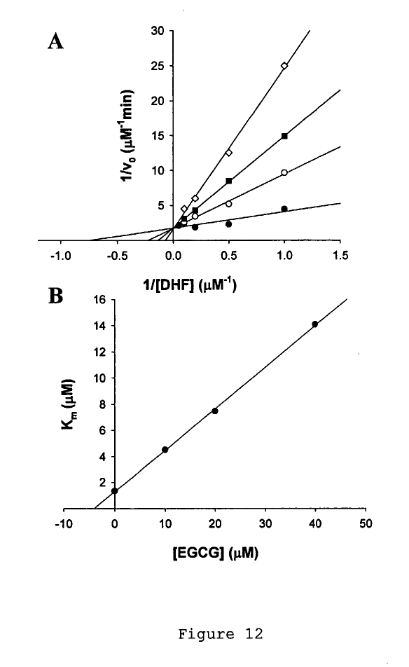

FIG. 12 shows (A) Lineweaver-Burk plots of the reaction of S. maltophilia DHFR with DHF and NADPH in the presence of TMP. TMP concentrations were (●) 10, (◯) 20, (▪) 40, and (⋄) 60 μM. Each point represents the mean±SD of five separate experiments. (B) Secondary plots for the apparent Michaelis constant of DHFR for DHF (Kₘ^{DHF}) versus the concentration of TMP.

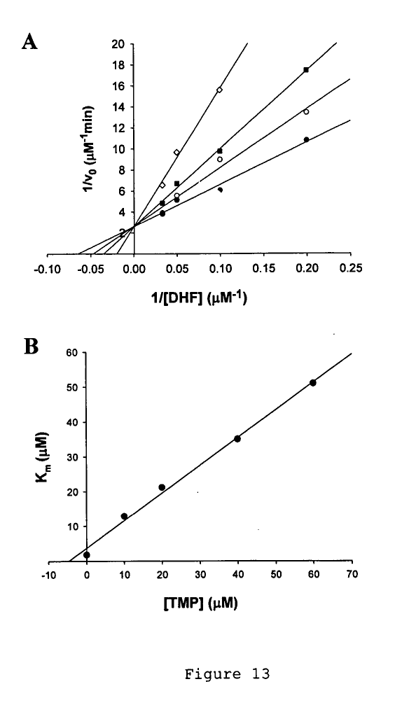

FIG. 13 shows (A) Lineweaver-Burk plots of the reaction of S. maltophilia DHFR with DHF and NADPH in the presence EGCG. EGCG concentrations were (●) 0, (◯) 10, (▪) 20, and (⋄) 40 μM. Each point represents the mean±SD of five separate experiments. (B) Secondary plots for the apparent Michaelis constant of DHFR for DHF (Kₘ^{DHF}) versus the concentration of EGCG.

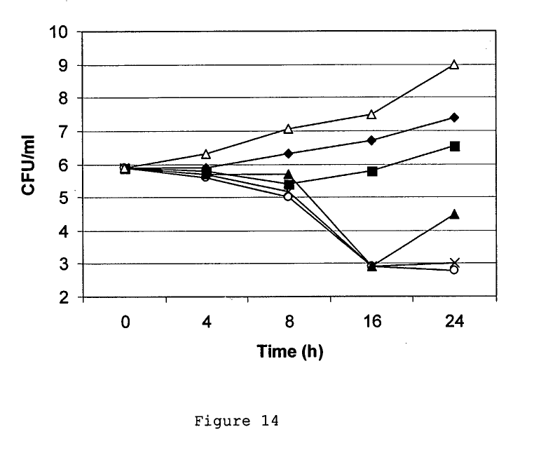

FIG. 14 shows the synergistic effects on S. maltophilia strain 1 with combinations of SMZ and EGCG. Control with no addition of antibiotics (Δ); EGCG at 16 μg/ml (⋄); SMZ at 512 μg/ml (▪). Combinations of SMZ/EGCG (μg/ml) 512/16. (∘); 256/16 (x); and 128/16 (▴).

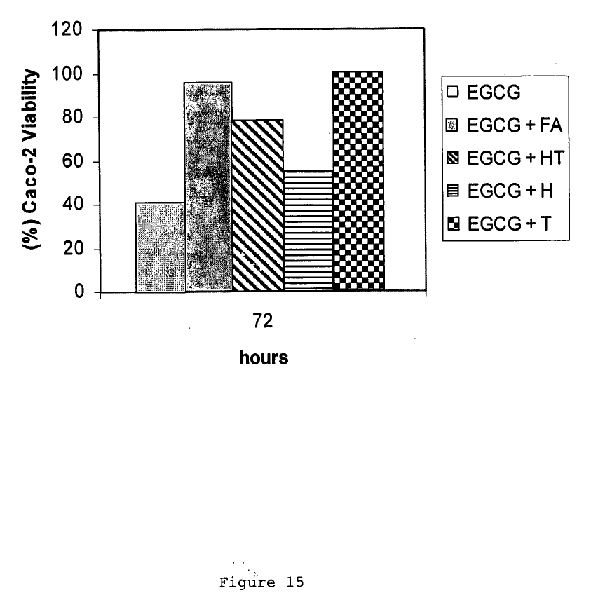

FIG. 15 shows DHFR inhibition by EGCG in colon cancer cells (Caco-2). Effect of EGCG (40 μM) on Caco-2 viability and reversion by folinic acid (FA), hypoxanthine-thymine (HT), hypoxanthine (H) and thymine (T) after 72 hours of treatment.

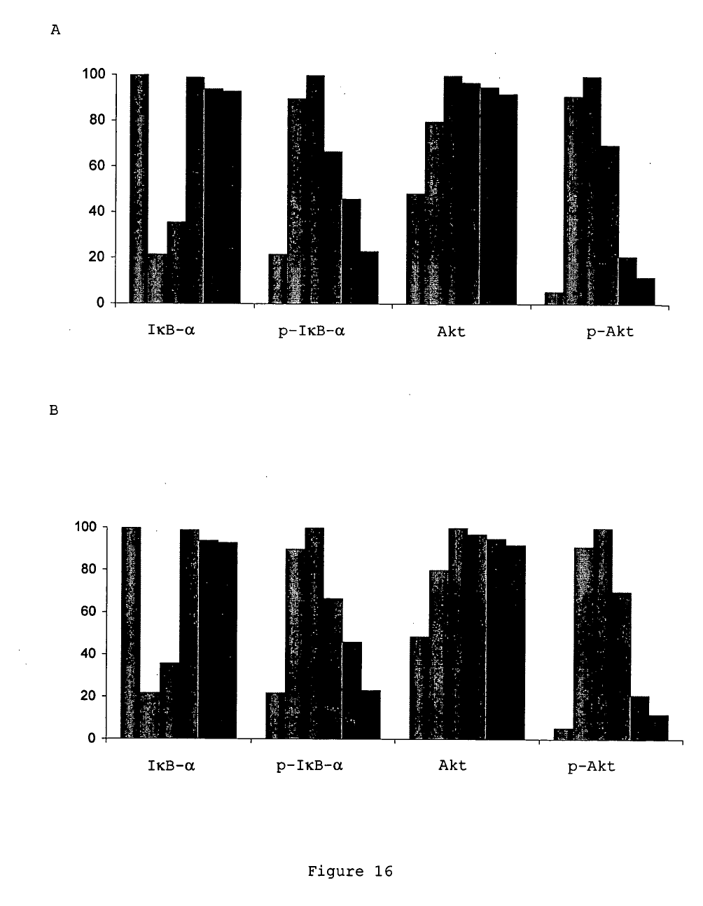

FIG. 16 shows (A) the effect of EGCG on TNF-α-mediated activation of NF-κB through degradation of IκBα and phosphorylation of Akt. (B) the effect of folinic acid. Bars show -TNFα, 0 μM EGCG; +TNFα, 0 μM EGCG; +TNFα, 1 μM EGCG; +TNFα, 20 μM EGCG; +TNFα, 40 μM EGCG; +TNFα, 80 μM EGCG. Relative Density normalized to β-Actin is shown on the Y-axis.

Table 1 shows a comparison of the inhibition by methotrexate, trimethoprim and EGCG of dihydrofolate reductase activity.

Table 2. Comparison of inhibition by MTX, TMP and EGCG of DHFR from S. maltophilia

Methods

Bacterial Strains

Eighteen strains of cotrimoxazole susceptible S. maltophilia were collected during the last year at the Hospital Universitario Virgen de la Arrixaca (Murcia, Spain) from clinical isolates. Bacteria were frozen at −70° C. in glycerol-meat medium and inoculated onto Columbia agar (Fluka Chemie GmbH, Madrid, Spain) supplemented with 5% of defibrinated sheep blood 48 and 24 h prior to susceptibility testing.

EGCG and Antibiotics

EGCG was obtained from Sigma Chemical Co. (Madrid, Spain). Stock dilutions were prepared on 0.15 mM H₃PO₄ to avoid oxidation of the drug. Other antibiotics were also obtained from Sigma. Stock dilutions of SMZ and TMP were prepared following the National Committee for Clinical Laboratory Standards (NCCLS) guidelines (30).

DHFR Modelling

Four different compounds from human and chicken DHFR crystal structures were used to define a binding envelope, shown in cyan; these were placed in a common orientation by superimposing backbone atoms from a common set of protein residues located around the compounds. Compounds from the following PDB structure files were used; 1DR1 (biopterin), 1S3V (TQD), 1S3W, and 1DLR. FIG. 2 was prepared using ViewerLite software²⁸.

Kinetic Analysis of DHFR Inhibition

DHF was obtained from Aldrich Chemie GmbH (Madrid, Spain) and NADPH from Sigma.

The activity of DHFR was determined at 25° C. by following the decrease of NADPH and DHF by absorbance measurements at 340 nm (ε=11,800 M⁻¹ cm⁻¹) by using a Perkin-Elmer Lambda-2 spectrophotometer with cuvettes of 1.0 cm light-path.

Temperature was controlled at 25° C. using a Haake D1G circulating bath with a heater/cooler and checked using a Cole-Parmer digital thermometer with a precision of ±0.1° C.

Experiments were performed at pH 7.58 and 25° C. in a buffer containing 2-(N-morpholino)ethanesulfonic acid (MES, 25 mM), sodium acetate (25 mM), tris(hydroxymethyl)aminomethane (Tris 50 mM), and NaCl (100 mM). To prevent the oxidation of catechins, the reaction mixture contained 1 mM ascorbic acid. The pH of the reaction was measured before and after of the experiment. The assays were started by the addition of enzyme. In the absence of the enzyme, the rate of change of absorbance was negligible.

Lineweaver-Burk plots were made of the reaction of chicken liver DHFR (3.3 nM) with DHF and NADPH. For initial velocity inhibition experiments one substrate (NADPH) was held constant at saturating concentration (100 μM) while the concentration of the other substrate (DHF) and the inhibitor (EGCG) were varied (FIG. 3). Experiments for the inhibition of DHFR by EGCG and ECG were performed in the presence of a saturating concentration of NADPH (100 or 200 μM) at pH 7.58. This initial concentration of saturating NADPH was considered as constant after the consumption of 10 μM DHF by the addition of enzyme. Data were fitted by nonlinear regression to the integrated form (31) of the Michaelis equation. The fitting was performed by using Marquart's algorithm (32) implemented in the Sigma Plot 2.01 for Windows (33).

Secondary plots for the apparent Michaelis constant of DHFR for dihydrofolate (K_{DHF}) versus the concentration of EGCG, were obtained from the Lineweaver-Burk plots (FIG. 3).

Progress curves for the slow, tight-binding inhibition of bovine liver DHFR by EGCG were determined by continuously monitoring the disappearance of NADPH and DHF after initiation of the reaction by the addition of 3.3 nM enzyme. Reaction mixtures contained buffer, NADPH (100 μM), DHF (20 μM) and EGCG. Assuming that the concentration of free inhibitor is not substantially altered by the formation of an enzyme-NADPH-inhibitor complex, the progress curve for the inhibition in the presence of saturating NADPH is described by Eqn. (1):
P=vₛt+(v₀−vₛ)(1−exp(−k′t))/k′

where vₛ, v₀ and k′ represent the steady-state velocity, initial velocity and apparent first-order rate constant, respectively.

The values of k′ at different EGCG concentrations were obtained by non-linear regression analysis of the progress curves. K_{I} and K_{I}* denote the respective dissociation constants for the initial and equilibrium binding of inhibitors to the enzyme-NADPH complex (FIG. 5).

Values for the inhibition of DHFR from different sources by methotrexate and trimethoprim have been obtained from the literatures

EGCG Cytotoxicity

L1210 cells were plated at a density of 10,000 cells/ml in 96-well plates with a “standard folate” RPMI 1640 medium supplemented with 10% FCS. Reversion experiments were carried out in an HT medium and/or by adding 50 μM ascorbic acid (AA). Cell injury was evaluated by a calorimetric assay for mitochondrial function using the MTT test.²⁹

The effect of different EGCG concentrations on L1210 cell growth after 29 h of treatment at two different folic acid concentrations was determined. The cells were previously adapted over a period of 2-3 days to grow in folate-free RPMI 1640 medium supplemented with 10% dialyzed FCS and 2 mM glutamine. This medium is subsequently referred to as LF (low folate). For the cytotoxicity assay, the cells were grown in LF media supplemented with folic acid (3 μM or 300 nM). Treatments were carried out in triplicate, and each experiment performed at least twice. Cell growth was determined by two different methods, Coulter counter Z₂ and hemocytometer (FIG. 7). IC₅₀ values were determined at each folic acid concentration added to the medium. IC₅₀ values were defined as the EGCG concentration that gave a 50% decrease in cellular growth compared with values of untreated control cells.

The growth-inhibitory effects of 10 μM EGCG at various folic acid concentrations were determined at two different time intervals. L1210 cells were cultured in a LF medium supplemented with the corresponding folic acid concentration. The data were obtained by using a Coulter counter and hemocytometer and expressed assuming a zero percentage of growth inhibition for untreated control (FIG. 8). Cells were maintained in the corresponding medium with 100 μg/ml of penicillin and streptomycin at 37° C. in a humid 7.5% CO₂, 95% air environment for all the experiments.

Purification of DHFR

For the DHFR extraction, S. maltophilia strain number 1, was inoculated onto fresh agar 24 h before using. Then, liquid medium was inoculated with the strain and these broth cultures were incubated at 37° C. and shaken at 100 cycles per min. Solid medium was MacConkey agar (Oxoid Ltd., Basingstoke, England). Liquid medium was Brilliant Green Bile 2% broth (Oxoid). Bacteria were grown to mid-log phase, harvested by centrifugation (1,600 rpm 30 min) and washed twice in 50 mM phosphate buffer (pH 7.0) followed each time by a new centrifugation (1,600 rpm 5 min). Cell lysis, centrifugation, and dialysis were carried out between 4 and 8° C. Fast protein liquid chromatography (FPLC) purification steps were performed at room temperature. Cell paste from 2 litres of culture was suspended in 30 ml of buffer A (5 mM Tris-HCl, pH 7.4, 1 mM EDTA) containing a protease inhibitor cocktail (5 μM pepstatin A, 1.5 μM bestatin, 0.1 mM phenylmethylsulfonyl fluoride, 1 mM benzamidine, and 1 mM 1,10-phenanthroline), and the cell suspension was subjected to homogenization in a Potter homogenised followed by ultrasonication. After centrifugation at 36,000 rpm for 30 min to remove cell debris, the supernatant was filtered. This supernatant was brought to 40% saturation with solid ammonium sulfate under continuous stirring. After 1 h the solution was centrifuged at 35,000 rpm for 30 min and the pellet discarded. Additional ammonium sulfate was added to the clear supernatant to give 90% saturation and stirred for 1 h. After centrifugation, the precipitates were suspended in 2 ml of buffer B (10 mM potassium phosphate buffer, pH 7.4, 2 mM β-mercaptoethanol). Concentrated enzyme (2 ml samples) was loaded onto a 26/60 gel-filtration column (Sephacryl S-75 16/60 Hi-Prep, Amersham Pharmacia Biotech Europe GmbH, Barcelona, Spain), equilibrated with buffer B and eluted at 0.5 ml/min. The active fractions were concentrated and stored at −80° C. until used.

DHFR Inhibition Experiments

Initial velocity inhibition experiments were carried out for the inhibition studies of S. maltophilia with TMP and EGCG. One substrate (NADPH) was held constant at saturating concentration while the other substrate (DHF) and the inhibitor (TMP or EGCG) were varied. To prevent the oxidation of EGCG the reaction mixture contained 1 mM N-acetylcysteine (Sigma). For MTX inhibition the slow development of EGCG inhibition was determined by continuously monitoring the disappearance of NADPH and DHF after initiation of the reaction by the addition of DHFR. Reaction mixtures contained buffer, NADPH (100 μM), DHF (20 μM), and various concentrations of MTX. The extent of recovery of enzymatic activity following inhibition induced by preincubation with DHFR inhibitors was determined as follows. DHFR was preincubated for 30 min at 25° C. in the buffer mixture containing MTX, TMP or EGCG. An aliquot of the incubation mixture was then diluted 500-fold into a reaction mixture containing buffer mixture, NADPH (100 μM), and DHF (20 μM). Recovery of enzyme activity was followed by continuous monitoring at 340 nm.

Broth Dilution MIC Determination

MICs for the 18 strains were determined by the broth dilution method at a final inoculum of 5×10⁵ CFU/ml, according to the guidelines recommended by the NCCLS by using cation-adjusted Mueller-Hinton broth (Fluka). After incubation at 37° C. for 24 h, the lowest concentration of the two-fold serially diluted EGCG at which no visible growth occurred was defined as its MIC.

Time Kill Assays for Detection of EGCG Bactericidal and Bacteriostatic Effects

A time kill assay was performed for strain 1. Glass tubes containing cation-adjusted Mueller-Hinton broth, with doubling antibiotic concentration were inoculated with 5×10⁵ CFU/ml and were incubated at 37° C. for 24 h. Antibiotic concentrations were chosen to comprise three doubling dilutions above and two doubling dilutions below the broth dilution MIC. Inoculation of each serially diluted antibiotic tube was performed following NCCLS guidelines for broth dilution method. Viability counts of antibiotic-containing suspensions were carried out at 0, 3, 6, 12 and 24 h, by plating 10 μl aliquots of 10-fold dilutions from each tube in sterile saline onto Columbia agar supplemented with 5% of defibrinated sheep blood. The plates used to recover organisms were incubated for up to 24 h. The lower limit of sensibility of colony counts was 100 CFU/ml. Time kill assay results were analyzed by determining changes in the log₁₀ CFU/ml compared to the counts at zero-time for the six different concentrations of EGCG. Bactericidal effect was defined at the lowest concentration that reduced the original inoculums by ≧3 log₁₀ CFU/ml after a period of time and bacteriostatic if the inoculums was reduced by 0 to 3 log₁₀ CFU/ml.

Checkerboard Synergy Testing

Checkerboard tests were performed for strain 1 by broth dilution in Mueller-Hinton broth combining eight doubling concentrations of EGCG with other eight dilutions of SMZ and TMP, respectively. Inoculum was prepared by suspending growth from blood agar plates in sterile saline to a final density of 0.5 McFarland and diluted in Mueller-Hinton broth to a final inoculum of 5×10⁵ CFU/ml. Tubes were incubated overnight aerobically at 37° C. Fractional inhibitory combinations (FICs) were calculated as the MIC of antibiotic and EGCG in combination divided by the MIC of the antibiotic or EGCG alone, and the FIC index was obtained by adding the FIC values. FIC indices were defined as synergic when values were ≦0.5, and antagonistic when values were >4. The results between synergy and antagonistic tendency were defined as additive or indifferent.

Time Kill Synergy Determinations

Strain 1 was tested by the time-kill method as described above. SMZ and EGCG were tested alone and in synergic combinations previously detected by the checkerboard method. The same combinations of SMZ and TMP were tested to compare with SMZ-EGCG. Viability counts were performed at 0, 4, 8, 16, and 24 h. Synergy was defined as ≧2 log₁₀ CFU/ml decrease in viable count with the combination at 24 h compared to the viable count with the more active of the two compounds alone.

Results

Steady-state kinetic data showing the inhibition by EGCG of DHF reduction with chicken liver DHFR are shown in FIG. 2.

A K_{I} (10.3 μM) for EGCG as a competitive inhibitor of DHF calculated from the secondary plot (FIG. 2b) was compared in Table 1 with values for methotrexate (1.3 nM) and trimethoprim (3.5 μM). Preincubation of the enzyme (1.65 μM) with EGCG (20 to 50 μM) for 30 min, followed by a 500-fold dilution into the standard DHF/NADPH assay did not produce any measurable inhibition. Thus the inhibition shown in FIGS. 2 and 3 must involve rapid reversible binding of EGCG to chicken liver DHFR.

However, EGCG acted as a slow tight-binding inhibitor of bovine liver DHFR (FIG. 4). In the absence of EGCG, the steady-state velocity of DHF reduction is rapidly established and only shows a minor deviation from linearity over a 15 min period due to substrate (DHF) depletion. In the presence of EGCG, a time-dependent decrease in the reaction rate which varies as a function of the inhibitor concentration is clearly apparent in FIGS. 4 and 5. Further evidence for slow-binding inhibition was obtained by adding aliquots of pre-incubation mixtures of EGCG and the bovine liver enzyme to substrate-containing assay mixtures. The resulting progress curves displayed time-dependent increases in the reaction rate and reached a steady-state velocity identical to that obtained without pre-incubation. The velocity then decreased due to the onset of substrate limitation. Such behaviour can be described by a mechanism that involves the rapid binding of the inhibitor (EGCG) to the enzyme (DHFR) to form an EI-complex which then undergoes a slow isomerization to form an EI* complex. Such a mechanism of inhibition of DHFR has been previously reported for folate analogues such as methotrexate and deazafolates^{15,16}. A complete kinetic analysis of the inhibition of bovine liver DFHR yielded the kinetic parameters given in Table 1.

Although ECG was also a potent inhibitor, polyphenols lacking the ester bonded gallate moiety (i.e. EGC and EC), did not inhibit bovine DHFR activity. These results indicate that the ester bonded gallate moiety is essential for potent inhibition of bovine liver DHFR.

Furthermore, a green tea extract containing significant amounts of EGCG also strongly inhibited the DHFR activity of both the bovine and chicken liver enzymes. Methotrexate is a stronger inhibitor (K_{I}* picomolar range) than EGCG (K_{I}* nanomolar range) for the two DHFRs studied. EGCG is therefore one of a class of “soft” DHF analogue inhibitors of DHFR¹⁷. Such compounds have several advantages in the treatment and prevention of cancer because they can attenuate the adverse side-effects often associated with DHFR inhibitors such as methotrexate that are currently in clinical use.

Further evidence for EGCG binding to the same site on DHFR as methotrexate comes from the pH dependence of the K_{I}* for the bovine liver enzyme. The low K_{DHF} value necessitated the use of the integrated Michaelis equation for the kinetic analysis of these data¹⁸. Although the DHFR catalysed reaction has been shown to occur via a random mechanism^{15,19}, under certain conditions ([NADPH]>>[DHF]) the reaction can be simplified to an ordered mechanism. A plot of V/K_{DHF} values against pH yielded a single pKₐ value of 8.7±0.2. which is similar to that reported for the human enzyme¹⁶. This has been interpreted in the case of the human enzyme as a shift of the pKₐ of Glu-30 from an intrinsic value of 5.6 to an observed value of about 8.7 on DHF binding. The similarity of the two pH profiles for the bovine and human enzymes suggests that the same ionizing residue is involved in catalysis. As Glu-30 is the only acidic residue at the pterin subsite of both enzymes^{16,20} it is considered to be the source of the proton for the reduction of DHF¹⁶.

The pH dependence of K_{I}* for EGCG binding to bovine DHFR gave a PKₐ value of 6.5±0.12, which is assigned to the Glu-30 which is conserved in mammalian DHFRs. This provides further evidence for EGCG binding at the same site as DHF. A similar pKₐ shift was assigned to Glu-30 for methotrexate binding to human DHFR¹⁶ which is readily explained by (i) the high degree of sequence homology between the bovine and human enzymes, (ii) the published structure of the human enzyme with methotrexate bound²¹ and (iii) our modelling of EGCG into the active site of human DHFR described below.

On searching the available compound-bound human DHFR structures in the Protein Data Bank (PDB)²², we identified a 1.8 Å structure (PDB accession code 1S3V²³) containing a tetrahydroquinazoline antifolate compound, TQD (FIG. 1), as the best available structural match for EGCG. Using the position of TQD as a guide, EGCG was docked into this protein structure and the EGCG-protein composite was then energy minimised using Insight II software.²⁷

Comparison with a range of other DHFR structures containing folate or various inhibitors showed that most of the EGCG lies within the consensual substrate/inhibitor envelope, with the exception of the non-ester trihydroxybenzene moiety. In order to accommodate this ring, the Leu-22 sidechain is required to adopt a different orientation; a precedent for this movement is provided by the crystal structure of a Tyr-22 mutant, which displays a similar geometry at this residue²⁴. Although folate, TQD, methotrexate and EGCG are significantly different in terms of their structural formulae, they have similar 3D shapes, and this appears to be an important determinant of their binding to DHFR. There are also specific hydrogen bonding interactions, most notably that involving Glu-30. For folate and methotrexate, adjacent heterocyclic and amino nitrogens of the compound form a pair of hydrogen bonds with the two oxygens of the Glu-30 sidechain (both O . . . N distances≦2.8 Å). In contrast, EGCG has only a single phenolic OH group available for hydrogen bonding to Glu-30 (O . . . O distance 2.7 Å). This is consistent with the pKₐ data discussed above. Other EGCG-protein contacts are similar to those found for TQD.

To determine whether EGCG inhibits DHFR activity in vivo, a mouse lymphoma cell line (L1210) was incubated with various concentrations of EGCG in a RPMI 1640 medium supplemented with 10% fetal calf serum (FCS). EGCG significantly inhibited L1210 growth in a concentration-dependent manner (IC₅₀=20 μM). If this was specifically due to inhibition of DHFR activity by EGCG, cell growth should be restored in a medium containing hypoxanthine-thymidine (HT medium). Antifolates block the de novo biosynthesis of thymine, purines and pyrimidines by inhibiting the synthesis of THF, an essential cofactor in these biosynthetic pathways. Cells that express hypoxanthine-guanine phosphoribosyl transferase (HGPRT), an enzyme essential for the recycling of purine nucleotides, can survive in the presence of antifolates in an HT medium. Control experiments showed that the inhibition of growth of L1210 cells by methotrexate was greatly attenuated in HT medium. FIG. 6 shows the time-dependent inhibition of L1210 growth by 20 μM EGCG. Although L1210 grown in HT medium showed a high level of inhibition reversal (FIG. 6), complete reversal was not obtained after the second day of the experiment. This partial lifting of EGCG inhibition in an HT medium may be due to secondary effects of EGCG at the concentration used in this assay. EGCG has been reported to have pro-oxidant activity in several cell lines (e.g. hepatoma cells)⁴. The production of reactive oxygen species (ROS) has been associated with the inhibition of cancer cell growth by tea polyphenols⁴. The inhibition of L1210 growth by EGCG was partially lifted by the inclusion of the antioxidant ascorbic acid in the reaction medium (FIG. 6). Similar results were obtained by co-treating the cells with N-acetylcysteine (NAC) (a glutathione precursor and scavenger of ROS) or superoxide dismutase. Growing L1210 in a HT medium containing ascorbic acid (FIG. 6), NAC or SOD completely removed the inhibitory effect of EGCG. These data provide strong evidence that the major site of action of EGCG in vivo is DHFR.

Further evidence for in vivo inhibition of DHFR by EGCG is provided by experiments with lymphoma cells grown in a RPMI low folate medium (FIGS. 7 & 8). These experiments were designed to investigate whether folate depletion has an effect on the sensitivity to EGCG. Cancer cell lines in standard cell culture medium are exposed to relatively high folate levels compared to the folate levels in human plasma²⁵. Consequently, the concentration of folates in the culture medium could affect the extent of EGCG inhibition of cell growth. The concentrations of EGCG needed to inhibit L1210 growth in a LF medium were much lower than were needed in a standard folate medium (FIG. 7). In a LF medium (30 nM), it was possible to study the time-dependent inhibition of L1210 growth at a lower concentration of EGCG (1 μM). Under these more physiologically relevant growth conditions inhibition by EGCG was completely reversed in an HT medium. These data show that inhibition of DHFR activity could be the major mechanism of the antitumor action of EGCG at physiological concentrations of folate substrates and blood serum levels of EGCG.

Antibacterial Action of EGCG on S. maltophilia

The MICs of EGCG against eighteen S. maltophilia isolates presented a range of 4-256 μg/ml (FIG. 9). The MIC for the 50% of the strains (MIC₅₀) was calculated to be 32 μg/ml, while the MIC for the 90% of the isolates (MIC₉₀) was of 64 μg/ml. The bactericidal action of EGCG was also examined. FIG. 10 shows representative data obtained with strain 1 exposed to 16, 32, 64 (MIC), 128, 256, and 512 μg of EGCG per ml. Bacteriostatic and bactericidal effects were observed at 12 h at concentration equal to 2 and 4-times the MIC, respectively. Regrowth was observed with EGCG (64 μg/ml) after 12 h incubation.

In order to determine if the effect of EGCG on S. maltophilia was due to DHFR inhibition, S. maltophilia DHFR was purified, and kinetically characterised with respect to its substrates and its inhibition by EGCG in vitro and other classical antifolate compounds.

Kinetic and Inhibition Studies of S. maltophilia DHFR

The Kₘ values for both substrates, NADPH and DHF, were determined using the partially purified enzyme. Due to the very low Kₘ values, the integrated Michaelis equation was used for this calculation. The Kₘ for NADPH was calculated to be 12 μM while that for DHF was 1.8 μM at pH 8.0. These values are similar to the Kₘ values of DHFRs from other species (34).

When DHFR activity was continuously assayed after addition of enzyme to assay mixtures containing MTX, DHF and NADPH, the resulting progress curves displayed a time-dependent decrease in reaction rate and finally attained a steady state velocity which varies as a function of inhibitor concentration (FIG. 12A), indicating the slow establishment of an equilibrium between enzyme, inhibitor, and the enzyme-inhibitor complex. Thus, MTX acts as a slow-binding inhibitor. Further evidence for slow-binding inhibition was obtained by adding aliquots of preincubation mixtures of DHFR and MTX to substrate containing assay mixtures. The resulting progress curves displayed a time-dependent increase in reaction rate and reached a steady state velocity identical to the velocity obtained without preincubation.

The kinetic analysis of the reaction progress curves recorded at pH 8.0 for MTX concentrations of up to 20 nM revealed that the initial reaction velocities (v₀) were independent of inhibitor concentration (FIG. 11A). Furthermore, a replot of k_{obs} against the inhibitor concentrations (FIG. 11B) gave a straight line with no indication of saturation kinetics. Both features indicate a mechanism involving the direct formation of a slowly dissociating EI complex without the initial formation of a rapidly reversible enzyme-inhibitor complex.

Although the DHFR catalysed reaction has been shown to occur via a random mechanism (35, 36), it can be simplified to an ordered mechanism whenever [NADPH]>>[DHF]. Provided that the concentration of free inhibitor is not substantially altered by the formation of an enzyme-NADPH-inhibitor complex, the progress curve for the inhibition in the presence of saturating NADPH can be described by Eqn. 1 (as above):
P=vₛt+(v₀−vₛ)(1−exp(−k_{obs}t))/k_{obs} [1]
where vₛ, v₀ and k_{obs} represent the steady-state velocity, initial velocity and apparent first-order rate constant, respectively. The apparent first-order rate constant is related to the inhibitor concentration by Eqn. 2, where Kᵢ denotes the dissociation constants for the initial binding of MTX to the enzyme-NADPH complex.

$$\begin{matrix}{k}_{obs} = {k}_{2} + {k}_{2} [ I ] / [ {K}_{i} ( 1 + [ S ] / {K}_{m}^{DHF} ) ] & [ 2 ] \\ {K}_{i} = \frac{{k}_{2}}{{k}_{1}} & [ 3 ]\end{matrix}$$

Further evidence for this mechanism was obtained by plotting 1/(k_{obs}−k₂) vs 1/[I]. The resulting straight line passes through the origin which is consistent with mechanism A. A dissociation constant Kᵢ of 18 pM and a rate constant k₂ of 1.1×10⁻³ s⁻¹ were obtained from a fit of the data presented in FIG. 12B to Eqn. 2. A value for k₁ of 6.17×10⁷ M⁻¹s⁻¹ was calculated by using Eqn. 3.

Steady-state kinetic data showing the inhibition by TMP of DHF reduction with S. maltophilia DHFR are shown in FIG. 12. The calculated values of Kᵢ for TMP as competitive inhibitor of DHFR calculated from the secondary plot (FIG. 12B) are compared in Table 2 with values for MTX. Preincubation of the enzyme (1.65 μM) with TMP (20 to 50 μM) for 30 min, followed by a 500-fold dilution into the standard DHF/NADPH assay did not produce any measurable inhibition. Thus, the inhibition shown in FIG. 12 must involve reversible binding of TMP to S. maltophilia DHFR, consistent with the kinetic profiles shown in this figure.

Preincubation experiments of the enzyme in the presence of different concentrations of EGCG did not showed any effect on enzymatic activity. However, EGCG affected to the initial rate of DHFR in the presence of their substrates, NADPH and DHF. Lineweaver-Burk plots at saturating concentration of NADPH and variable DHF and EGCG showed a set of straight lines, which intercepts on the ordinate axis (FIG. 13A). These results are characteristic of a reversible and competitive inhibition respect to DHF with a calculated inhibition constant (Kᵢ) of 4.04 μM.

The results showed that EGCG is an effective inhibitor of S. maltophilia DHFR which follows a similar inhibition mechanism to TMP but differs from that of MTX.

A comparison of the kinetic parameters for inhibition of DHFR by EGCG with those of MTX and TMP is shown in Table 2. MTX is a stronger inhibitor (Kᵢ picomolar range) than EGCG or TMP (Kᵢ micromolar range) for S. maltophilia DHFR. Although MTX presents higher activity on S. maltophilia, its clinical use is precluded because it is also a strong inhibitor of human DHFR.

However, EGCG may represent a useful therapeutic for the treatment of S. maltophilia infections, especially in patients with low tolerance to TMP. It has been observed that high doses of TMP are difficult to tolerate for elderly patients with poor renal function, producing severe skin reactions, bone marrow suppression, and thrombocytopenia.

Comparative Activity of EGCG Combined with Other Agents

By using checkerboard titrations the synergy between TMP and sulfamide (cotrimoxazole) against S. maltophilia was confirmed (FIC=0.188). Checkerboard titrations also revealed significant synergism between EGCG and SMZ against S. maltophilia (FIC=0.25). No FIC indices indicating antagonism were observed for any of the combinations. Similar experiments between EGCG and TMP showed no synergic FIC indices (lowest FIC=0.625), indicating a purely additive behaviour between both drugs. Some drug combinations found synergic by checkerboard titrations were analyzed by the time kill curve method, also showing a synergic behaviour (FIG. 14). These results indicate that EGCG could act as an inhibitor of DHFR and that sulfonamide could increase the antibiotic effect of EGCG by decreasing the folic acid levels in the bacteria.

Inhibition of Human DHFR by Tea Catechins

Green tea extracts containing significant amounts of tea catechins strongly inhibited the activity of recombinant human DHFR (rHDHFR). In order to detect which components of these extracts were responsible of such inhibition, DHFR activity was assayed in the presence of EC, EGC, ECG or EGCG. The results showed that both ECG and EGCG were potent inhibitors of the human enzyme, however polyphenols lacking the ester bonded gallate moiety (e.g. EGC and EC) did not inhibit rHDHFR. These results provide indication that the ester bonded gallate moiety is important for inhibition of this enzyme as was determined above for the bovine enzyme. When DHFR activity was continuously assayed after addition of enzyme to assay mixtures containing ECG or EGCG and enzyme substrates (NADPH and DHF), the resulting progress curves displayed time-dependent decreases in the reaction rates and finally attained steady-state velocities which varied as a function of inhibitor concentration, providing indication that the slow establishment of a steady-state between enzyme, inhibitor, and the enzyme-inhibitor complex. Determination of Ki showed that this constant is in the low-micromolar range (Ki=2 μM).

Binding of EGCG to rHDHF

The binding of EGCG to free DHFR was determined by following the decrease in enzyme fluorescence that occurs on formation of the enzyme-inhibitor complex. When bovine liver DHFR fluorescence is excited at 290 nm its emission spectrum shows a maximum at 340-350 nm. The binding of EGCG quenches this fluorescence. The data for the resulting titration curves were used for dissociation constant determinations. The data showed that the dissociation constants of free rHDHFR for EGCG is 800 nM, in the range of the dissociation constant of bovine liver DHFR for this compound.

Inhibition of DHFR by EGCG in Human Cancer Cells

To determine whether EGCG inhibits DHFR activity in vivo, two human cancer cell, (Caco-2 from colon cancer and Jurkat T a lymphoma cell line) were incubated with various concentrations of EGCG in a standard medium. EGCG significantly inhibited the growth of both cancer cell lines in a concentration-dependent manner (IC₅₀₌₃₀ μM and 10 μM for Caco-2 and Jurkat T, respectively). Moreover, as other folate compounds, EGCG produced G0/G1 phase arrest of the cell cycle, and induction of apoptosis. All these effect were highly attenuated by growing the cells in HT medium and in the presence of folinic acid or thymine, indicating that the effects were due, mainly to DHFR inhibition (FIG. 15).

As was observed in a human esophageal squamous carcinoma cell line, KYSE 510 (Fang et al., 2003), p16 had hypermethylation status in Caco-2 and Jurkat T cells. We also observed that after 7-days treatment of these two cell cancer lines with 20 μM EGCG, the p16 methylation pattern changed from methylated to unmethylated. To determine whether the DNA hypomethylation induced by EGCG in these cancer lines was due to the inhibition of 5-cytosine DNA methyltransferase (DNMT) (Fang et al., 2003) or to reduction in the folate pool associated with DHFR inhibition, two series of experiments were performed.

First, we studied the methylation status of p16 in Caco-2 cells treated with MTX (a classical antifolate compound) and in Jurkat T cells grown in RPMI medium with low folate levels. In both cases, reversal of hypermethylation was observed, due, probably, to SAM depletion and methyltransferase SAH inhibition as a consequence of the disruption of the folate cycle in the cells. In a second series of experiments, we determined whether hypomethylation of p16 induced by low folate level or by treatments with EGCG or MTX could be reversed by growing the cells in a medium containing folinic acid. In all cases p16 was found to have a hypermethylation status.

Although EGCG has been shown to directly inhibit DNMT in vitro (Fang et al., 2003), the reversal of hypomethylation of p16 by folinic acid in cancer cells treated with EGCG provides indication that DNA hypomethylation observed in vivo is more related to the disruption of the folate cycle associated with DHFR inhibition, than to direct inhibition of DNMT. Many tumour suppressor and receptor genes have been reported to be hypermethylated and transcriptionally silenced during the development of different types of cancers (Widschwendter & Jones, 2002). The induction of low folate levels in cancer cells by EGCG treatment prevents the hypermethylation and silencing of these key genes and may contribute to the prevention of carcinogenesis.

The Anti-Inflammatory Properties of EGCG is Related with its Antifolate Activity.

To check if the antifolate activity of EGCG is related to the suppression of activation of NF-κB, cells were grown in the presence of EGCG or MTX. After 24 hours, the cells were induced with TNF-α and the content of proteins related to NF-κB activation (IκBα, phospho-IκBα, Akt and phosphor-Akt) was checked by Western blot (FIG. 16A). The effects of EGCG and MTX were highly reversed by folinic acid in both cases (FIG. 16B for EGCG). The data present here clearly shows that the prophylactic, anti-carcinogenic and anti-inflammatory properties of EGCG are related to its antifolate activity.

In conclusion, the data provided herein shows for the first time that gallated tea polyphenols act as DHFR inhibitors in vitro and in vivo, at concentrations usually found in the blood of tea drinkers. The “soft” character of such compounds may be developed for use in the prevention and treatment of cancer with significantly reduced side effects compared to those of the DHFR inhibitors currently in use in chemotherapy such as methotrexate. EGCG is advantageous in having differential effects on normal and cancer cells. Importantly, at physiologically attainable concentrations, EGCG kills cancer cells through apoptosis but has little or no effect on normal cells. Inhibition of DHFR by EGCG explains this differential effect and is entirely consistent with the observation that antifolate compounds are more active on cancer cells with a high-turnover of DNA. Our conclusions highlight the potential of gallated-polyphenols and their derivatives for clinical application as anti-carcinogenic and antibiotic agents and in the treatment of conditions such as psoriasis¹⁻³.

TABLE 1

| Enzyme | | K_{I} | k₅ | k₆ | k₅/k₆ | K_{I}* |
|---|---|---|---|---|---|---|
| Source | Inhibitor | (nM) | (min⁻¹) | (min⁻¹) | (ratio) | (pM) |
| Bovine | EGCG | 109 | 0.13 | 0.004 | 32.5 | 2900 |
| liver | ECG | 51.3 | 0.11 | 0.015 | 7.1 | 6550 |
| Chicken | EGCG | 10300 | | | | |
| liver | methotrexate | 1.3 | 2.9 | 0.020 | 150 | 9 |
| | trimethoprim | 3530 | | | | |
| E. coli | methotrexate | 3.6 | 6.9 | 0.026 | 270 | 14 |
| | trimethoprim | 0.49 | 2.0 | 0.086 | 23 | 20 |
| S. faecium | methotrexate | 23 | 5.1 | 0.013 | 390 | 59 |
| | trimethoprim | 4.6 | 2.1 | 0.58 | 3.6 | 1000 |

TABLE 2

| | Type of inhibition | |
|---|---|---|
| Compound | observed | Kᵢ (nM) |
| MTX | competitive (slow- | (0.018 ± 0.002) |
| | binding) | |
| TMP | competitive (reversible) | (4,690 ± 120) |
| EGCG | competitive (reversible) | (4,040 ± 100) |

### REFERENCES

1. Mukhtar, H. & Ahmad, N. Am. J. Clin. Nutr. 71, 1698S-1702S (2000).
2. Mabe, K. et al. Antimicrob. Agents Chemother. 43, 1788-1791 (1999).
3. Heyendael V. M. R. et al. N. Engl. J. Med. 349, 658-665 (2003)
4. Waltner-Law, M. E. et al J. Biol. Chem. 277, 34933-34940 (2002).
5. Jung, Y. D. et al Int. J. Exp. Path. 82, 309-316 (2001).
6. Gupta, S. et al Arch. Biochem. Biophys. 410, 177-185 (2003).
7. Dong, Z. et al. Cancer Res. 57, 4414-4419 (1997).
8. Lin, Y. L. et al. Mol. Pharmacol. 52, 465-472 (1997).
9. Suganuma, M. et al. Biofactors 13, 67-72 (2000).
10. Jung, Y. D., Kim, M. S., Shin, B. A., Chay, K. O., Ahn, B. W., Liu, W. et al Br. J. Cancer 84, 844-850 (2001).
11. Nam, S. et al. J. Biol. Chem. 276, 13322-13330 (2001).
12. Yang, C. S., Nature 389, 134-135 (1997).
13. Jankun, J. et al Nature 387, 561 (1997).
14. Gready, J. E. Adv. Pharmacol. Chemother. 17, 37-102 (1980).
15. Stone, S. R. et al. Biochim. Biophys. Acta 869, 275-285 (1986).
16. Williams, E. A. et al Biochemistry 31, 6801-6811 (1992).
17. Nordberg, G. Approaches to soft drug analogues of dihydrofolate reductase inhibitors. Design and synthesis. Acta Universitatis Upsaliensis. Comprehensive Summaries of Upssala Dissertations from the Faculty of Pharmacy 252, 75 pp, Uppsala (2001).
18. Cornish-Bowden, A. in Fundamentals of Enzyme Kinetics, pp 34-37, Butterworth and Co., London (1979).
19. Rod, T. H. et al Proc. Natl. Acad. Sci. USA 100, 6980-6985 (2003).
20. Baumann, H. et al. Eur. J. Biochem. 60, 9-15 (1975).
21. Cody, V. et al. Anticancer Drug Des. 7, 483-491 (1992).
22. Berman, H. M. et al. Nucleic Acids Research 28, 235-242 (2000)
23. Cody, V. et al. Acta Cryst. Sect. D 60, 646-655 (2004).
24. Lewis, W. S. et al J. Biol. Chem. 270, 5057-5064 (1995).
25. Backus, H. H. et al Int. J. Cancer 15, 771-778 (2000).
26. Correa, A. et al Ann. Epidemiol. 10, 476-477 (2000).
27. Insight II, release 2000.1 (2000), Accelrys Ltd.,
Cambridge CB4 0WN, UK.
28. ViewerLite release 5.0 (2002), Accelrys Ltd., Cambridge CB4 0WN, UK
29. Carmichael, J. et al Cancer Res. 47, 936-42 (1987)
30. National Committee for Clinical Laboratory Standards. 1997. Methods for dilution antimicrobial susceptibility test for bacteria that grow aerobically, 4^{th} ed. Approved standard. NCCLS publication no. M7-A4. National Committee for Clinical Laboratory Standards, Villanova, Pa.
31. Cornish-Bowden, A. 1979. In: Fundamentals of Enzyme Kinetics. p. 34-7. Butterworth and Co. London.
32. Marquardt, D. W. 1963. An algorithm for least-squares estimation of nonlinear parameters. J. Soc. Ind. Appl. Math. 11:431-441.
33. SPSS Inc. 2003. Sigma Plot SPSS Inc. Chicago, Ill.
34. Wilquet V et al 1998 Eur. J. Biochem. 255:628-637.
35. Rod, T. H. et al 2003. Proc. Natl. Acad. Sci. USA 100:6980-6985.
36. Stone, S. R. et al 1986 Biochim. Biophys. Acta 869:275-285.

1. A method of producing an anti-folate compound comprising;
providing an (−)-epigallocatechin gallate (EGCG) compound, and;
determining the interaction of said EGCG compound with dihydrofolate reductase (DHFR).

2. A method according to claim 1 wherein the EGCG compound comprises a gallate moiety.

3. A method according to claim 2 wherein the EGCG compound is a polyphenol.

4. A method according to claim 3 wherein the EGCG compound is a flavonoid.

5. A method according to claim 4 wherein the EGCG compound is a flavan-3-ol.

6. A method according to claim 5 wherein the EGCG compound is EGCG.

7. A method according to any one of claims 1 to 6 comprising;
modifying the structure of the ECGC compound, and;
determining the interaction of the modified EGCG compound with DHFR.

8. A method according to claim 7 wherein the interaction is determined in silico.

9. A method for producing an anti-folate compound comprising:
providing a structure comprising a three-dimensional representation of DHFR or a portion of DHFR;
providing an EGCG compound structure to be fitted to said DHFR structure or selected coordinates thereof
fitting the EGCG compound structure to said DHFR structure.

10. A method according to claim 9 wherein the EGCG compound structure is EGCG.

11. A method according to claim 9 or claim 10 comprising modifying the structure of said EGCG compound.

12. A method according to claim 11 wherein the structure of the EGCG compound is modified to improve or enhance interaction with the DHFR structure.

13. A method according to claim 11 or claim 12 comprising fitting the modified EGCG compound to the DHFR structure or selected coordinates thereof.

14. A method according to claim 13 comprising identifying a modified EGCG compound structure with improved or optimised properties.

15. A method according to claim 14 comprising identifying a modified EGCG compound structure with improved or optimised DHFR binding properties.

16. A method according to claim 14 or claim 15 comprising modifying or optimising the structure of the modified EGCG compound.

17. A method according to any one of claims 14 to 16 comprising obtaining or chemically synthesizing the modified EGCG compound.

18. A method of producing an anti-folate compound comprising:
(a) providing a three dimensional structure of a complex of DHFR and a starting EGCG compound;
(b) designing a modified EGCG compound using the three-dimensional structure; and,
(c) chemically synthesizing the modified EGCG compound.

19. A method according to claim 17 or claim 18 comprising contacting the modified EGCG compound with an isolated DHFR protein to determine the ability of said compound to interact with or inhibit the DHFR protein.

20. A method according to claim 17 or claim 18 comprising;
forming a complex of a DHFR protein and said modified EGCG compound; said complex diffracting X-rays for the determination of atomic coordinates of said complex; and
analysing the complex by X-ray crystallography to determine the interaction of said modified EGCG compound with DHFR.

21. A method according to any one of claims 1 to 8 or 19 to 20 wherein the interaction of the modified EGCG compound with DHFR is determined in vitro.

22. A method according to claim 21 wherein the interaction is determined by contacting the EGCG compound with DHFR, and; determining the binding of the EGCG compound to DHFR.

23. A method according to claim 22 wherein binding is determined relative to the binding of EGCG to DHFR.

24. A method according to any one of claims 21 to 23 wherein
the interaction is determined by contacting the EGCG compound with DHFR, and
determining the inhibition of DHFR activity by the EGCG compound.

25. A method according to any one of claims 21 to 24 wherein the EGCG compound is comprised in a library of EGCG compounds, said library being contacted with DHFR.

26. A method according to any one of claims 21 to 25 comprising identifying an EGCG compound which binds to DHFR and/or inhibits DHFR activity as a putative anti-folate compound.

27. A method according to claim 26 comprising modifying the identified EGCG compound to optimise its pharmaceutical properties.

28. A method according to any one of claims 7 and 17 to 27 comprising contacting the modified EGCG compound with a mammalian cell line and determining the effect of the compound on the cell.

29. A method according to claim 28 wherein the effect of the modified EGCG compound is determined relative to the effect of EGCG on said cell.

30. A method according to claim 28 or claim 29 wherein the apoptosis and/or cell cycle arrest in a cancer cell line is determined.

31. A method according to claim 28 or claim 29 wherein the expression of one or more biomarkers is determined.

32. A method according to claim 31 wherein said one or more biomarkers are selected from the group consisting of activator protein-1 (AP-1), nuclear factor-kappaB (NF-κB), tumor necrosis factor alpha (TNF-α), vascular endothelial growth factor (VEGF), nitric oxide synthase (NOS), urokinase, ornithine decarboxylase, matrix metalloproteinase and cyclooxygenase.

33. A method according to any one of claims 7 and 17 to 32 comprising determining the effect of the modified EGCG compound on tumour invasion and angiogenesis in a non-human animal model.

34. A method according to any one of claims 7 and 17 to 33 comprising determining the uptake, in vivo half-life, and/or toxicology of the modified EGCG compound in a non-human animal model.

35. A method according to any one of claims 7 and 17 to 34 comprising formulating the modified EGCG compound with a pharmaceutically acceptable carrier, adjuvant or excipient.

36. A modified EGCG compound which inhibits DHFR and which is identified or produced by a method according to any one of claims 7 and 17 to 35.

37. Use of a modified EGCG compound according to claim 36 in the manufacture of a medicament for the treatment of a disease condition.

38. Use according to claim 37 wherein said disease condition is cancer, an atherosclerotic disorder, a pathogen infection or an inflammatory disorder.

39. A method of treating a disease condition in an individual comprising administering a modified EGCG compound according to claim 36.

40. A method according to claim 39 wherein said disease condition is cancer, pathogen infection or an inflammatory disorder.

41. A method for preparing a pharmaceutical composition comprising:
(a) producing a modified EGCG compound by a method according to any one of claims 7 and 17 to 34;
(b) optimising the structure of the EGCG compound; and
(c) preparing a pharmaceutical composition containing the optimised EGCG compound.

42. A method according to claim 41 wherein the pharmaceutical composition is prepared by admixing the optimised EGCG compound with a pharmaceutically acceptable carrier, adjuvant or excipient.

43. A method of determining the optimal physiological dose of an EGCG compound for inhibiting growth of a cancer cell comprising:
determining the inhibition of DHFR in a cancer cell by progressively increasing doses of the EGCG compound;
determining the viability of a non-cancer cell in the presence of said progressively increasing doses; and
selecting the dose of the EGCG compound that achieves DHFR inhibition in the cancer cell with continued viability of the non-cancer cell.

44. The method according to claim 43, wherein the optimal physiological dose of the EGCG compound is determined in vitro.

45. The method according to claim 43, wherein the optimal physiological dose of the EGCG compound is determined in vivo.

46. The method according to any one of claims 43 to 45, wherein the EGCG compound is obtained from an extract of green tea.

47. The method according to any one of claims 43 to 45, wherein the EGCG compound is a synthetic EGCG compound.

48. The method according to any one of claims 43 to 47 comprising formulating the dose of the EGCG compound with a pharmaceutically acceptable carrier, adjuvant or excipient.

49. The method according to claim 48 further comprising formulating the dose of the EGCG compound and pharmaceutically acceptable carrier, adjuvant or excipient with an agent which modulates intracellular pH.

50. The method according to any one of claims 43 to 49, wherein the EGCG compound is EGCG.

51. A method for identifying and/or obtaining a putative anti-folate compound comprising:
contacting an EGCG compound and a test compound with DHFR; and
determining the binding of the EGCG compound to DHFR in the presence of the test compound,
wherein displacement of the binding of the EGCG compound to DHFR by the test compound indicates that the test compound is a putative anti-folate compound.

52. A method according to claim 51, wherein the EGCG compound is radiolabelled.

53. A method according to claim 51 or claim 52, wherein the EGCG compound is EGCG.

54. An anti-folate compound identified by the method of any one of claims 51 to 53.

55. A composition comprising an EGCG compound and an agent which modulates intracellular pH, for use in the treatment of a disease condition.

56. Use of an EGCG compound and an agent which modulates intracellular pH in the manufacture of a medicament for use in the treatment of a disease condition.

57. A method of treating a disease condition comprising administering an EGCG compound and an agent which modulates intracellular pH.

58. A method of making a composition for use in the treatment of a disease condition comprising:
admixing an EGCG compound, an agent which modulates intracellular pH and a pharmaceutically acceptable excipient.

59. A composition according to claim 55, a use according to claim 56 or a method according to any one of claims 57 to 58 wherein the EGCG compound is EGCG.

60. A composition, use or method according to any one of claims 55 to 59 wherein the agent increases the intracellular pH of prokaryotic cells.

61. A composition, use or method according to claim 60 wherein the agent is a proton pump inhibitor.

62. A composition, use or method according to claim 61 wherein the proton pump inhibitor is selected from the group is selected from the group consisting of omeprazole, lansoprazole, pantoprazole, rabeprazole and esomeprazole.

63. A composition, use or method according to any one of claims 60 to 62 wherein the disease condition is chronic gastritis, peptic ulceration, gastric cancer and Helicobacter pylori infection.

64. A composition, use or method according to any one of claims 55 to 59 wherein the agent increases the intracellular pH of eukaryotic cancer cells.

65. A composition, use or method according to claim 64 wherein the agent is cesium chloride, rubidium chloride or potassium chloride.

66. A composition, use or method according to claim 64 or claim 65 wherein the disease condition is cancer.

67. A method of treating a cancer cell comprising treating the cancer cell to increase the intracellular pH of said cancer cell and administering an EGCG compound in an effective amount to inhibit DHFR in said cell.

68. A composition comprising an EGCG compound and an agent which inhibits bacterial folic acid synthesis.

69. Use of an EGCG compound and an agent which inhibits bacterial folic acid synthesis in the manufacture of a medicament for use in the treatment of a disease condition.

70. A method of treating a disease condition comprising administering an EGCG compound and an agent which inhibits bacterial folic acid synthesis.

71. A method of making a composition for treating a disease condition comprising:
admixing an EGCG compound, an agent which inhibits bacterial folic acid synthesis and a pharmaceutically acceptable excipient.

72. A composition according to claim 68, a use according to claim 69 or a method according to any one of claims 70 or 71 wherein the EGCG compound is EGCG.

73. A composition according to claim 68, a use according to claim 69 or a method according to any one of claims 70 or 71 wherein the agent which inhibits bacterial folic acid synthesis is an inhibitor of the incorporation of p-aminobenzoic acid

74. A composition, use or method according to claim 73 wherein the inhibitor of p-aminobenzoic acid incorporation is an amide of a sulfonic acid or sulphonamide.

75. A composition, use or method according to claim 74 wherein the amide of a sulfonic acid is sulfamethoxazole.

76. A composition, use or method according to claim 74 wherein the sulphonamide and the EGCG compound are formulated to produce a 20:1 ratio of sulphonamide to EGCG compound in blood and tissues of an individual upon administration.

77. A composition, use or method according to claim 74 wherein the sulphonamide and the EGCG compound are formulated in the ratio of 5:1 of sulphonamide to EGCG compound.

78. A composition, use or method according to any one of claims 69 to 77 wherein the disease condition is a bacterial infection.

79. A composition, use or method according to claim 78 wherein the bacterial infection is a Stenotrophomonas maltophilia infection.

80. A composition, use or method according to any one of claims 69 to 77 wherein the condition is selected from the group consisting of Pneumocystis carinii pneumonia, typhoid fever, shigellosis, enterotoxigenic Escherichia coli diarrhoea, Nocardia infection, otitis media, and chronic bronchitis.

81. A composition comprising an EGCG compound and an anti-folate compound.

82. Use of an EGCG compound and an anti-folate compound in the manufacture of a medicament for use in the treatment of a disease condition.

83. A method of treating a disease condition in an individual comprising administering an EGCG compound and an anti-folate compound.

84. A method of making a composition for use in treating a disease condition comprising:
admixing an EGCG compound, an anti-folate compound and a pharmaceutically acceptable excipient.

85. A method for enhancing the efficacy of an anti-folate treatment which comprises co-administering said anti-folate with an effective amount of an EGCG compound to achieve inhibition of DHFR.

86. The composition of claim 81, use of claim 82 or method of claims 80 to 85 wherein the anti-folate is selected from the group consisting of methotrexate, trimethoprim (TMP), sulfamethoxazole (SMZ) and cotrimoxazole (TMP-SMZ).

87. A composition comprising an EGCG compound in combination with an agent that decreases folic acid intestinal absorption or decreases the transport of folic acid to cells.

88. Use of an EGCG compound and an agent that decreases folic acid intestinal absorption or decreases the transport of folic acid to cells in the manufacture of a medicament for use in the treatment of a disease condition.

89. A method of treating a disease condition comprising administering an EGCG compound in combination with an agent that decreases folic acid intestinal absorption or decreases the transport of folic acid to cells.

90. A method of making a composition for use in treating a disease condition comprising:
admixing an EGCG compound, an agent that decreases folic acid intestinal absorption or decreases the transport of folic acid to cells, and a pharmaceutically acceptable excipient.

91. A composition according to claim 87, a use according to claim 88 or a method according to claim 89 or claim 90 wherein the EGCG compound is EGCG.

92. A composition according to claim 87, a use according to claim 88 or a method according to claim 89 or claim 90 wherein the agent that decreases folic acid intestinal absorption or decreases the transport of folic acid to cells is omeprazole, ranitidine or sulfasalazine.

93. Use of an EGCG compound in the manufacture of a medicament for use in the treatment of bacterial infection in an individual with low tolerance to trimethoprim (TMP).

94. A method of treating a bacterial infection in an individual with low tolerance to trimethoprim (TMP) comprising administering an EGCG compound.

95. A method of making a composition for use in treating a bacterial infection in an individual with low tolerance to trimethoprim (TMP) comprising:
admixing an EGCG compound and a pharmaceutically acceptable excipient.

96. A use according to claim 93 or a method according to claim 94 or claim 95 wherein the EGCG compound is EGCG.

97. A use according to claim 93 or a method according to claim 94 or claim 95 wherein bacterial infection is a Stenotrophomonas maltophilia infection.
